(19) United States
(12) Patent Application Publication
(10) Pub. No.: US 2023/0338419 A1
(43) Pub. Date: Oct. 26, 2023

(54) METHODS AND APPARATUS FOR DIAGNOSIS AND DETECTION OF CARDIAC ALLOGRAFT VASCULOPATHY

(71) Applicants: The Trustees of the University of Pennsylvania, Philadelphia, PA (US); Case Western Reserve University, Cleveland, OH (US)

(72) Inventors: Eliot Peyster, Philadelphia, PA (US); Andrew Janowczyk, East Meadow, NY (US)

(73) Assignees: The Trustees of the University of Pennsylvania, Philadelphia, PA (US); Case Western Reserve University, Cleveland, OH (US)

(21) Appl. No.: 18/186,445

(22) Filed: Mar. 20, 2023

Related U.S. Application Data
(60) Provisional application No. 63/322,007, filed on Mar. 21, 2022.

(51) Int. Cl. A61K 35/12; A61L 27/38

(52) U.S. Cl. CPC A61K 35/12; A61L 27/38

(57) ABSTRACT

Provided herein are methods, systems and apparatus for diagnosing, predicting and/or preventing acute allograft vasculopathy in an organ transplant patient. The method comprises executing on a processor the steps comprising: analyzing clinical risk factors of a cardiac, kidney or liver transplant patient in a model which distinguishes between clinical biomarkers of allograft vasculopathy patients and non-allograft vasculopathy patients, and analyzing digital biopsy images from the transplant patient to morphologically differentiate patients which are prE allograft vasculopathy or which have active allograft vasculopathy from patients without acute allograft vasculopathy; assigning prE-, active or non-allograft vasculopathy status to a patient based on the outcome of the analysis of the clinical biomarkers and morphologic biomarkers; and where preE or active statis is assigned, treating the patient.

### STATEMENT OF FEDERALLY SPONSORED RESEARCH

This invention was made with government support under U01 CA239055-01 awarded by National Institutes of Health. The government has certain rights in this invention. This invention was made with government support under KL2TR001879 by National Institutes of Health. The government has certain rights in this invention. U01 CA239055-01 was awarded by the National Cancer Institute. KL2TR001879 was awarded by the National Center for Advancing Translational Sciences.

### BACKGROUND OF THE INVENTION

Heart transplantation remains the first line treatment for eligible patients with end-stage heart disease. Each year, more than 4000 heart transplants are performed worldwide [Lund, L.H., et al., The registry of the International Society for Heart and Lung Transplantation: thirty-first official adult heart transplant report--2014; focus theme: retransplantation. J Heart Lung Transplant, 2014. 33(10): p. 996-1008], offering markedly improved quality of life and longevity for the vast majority of recipients. However, transplanted hearts do not last forever, with recipient immune responses against the allograft posing a continuous threat that requires vigilant surveillance and careful medical management. While acute episodes of allograft rejection are the most apparent mechanism of allo-immune injury experienced by transplant recipients, indolent, immune-mediated vascular injury, known as cardiac allograft vasculopathy (CAV), represents the leading cause of allograft failure after the first-year post-transplant [Lund, L.H., et al., cited above; Bruneval, P., et al., The XIIIth Banff Conference on Allograft Pathology: The Banff 2015 Heart Meeting Report: Improving Antibody-Mediated Rejection Diagnostics: Strengths, Unmet Needs, and Future Directions. Am J Transplant, 2017. 17(1): p. 42-53; Chih, S., et al., Allograft Vasculopathy: The Achilles’ Heel of Heart Transplantation. Journal of the American College of Cardiology, 2016. 68(1): p. 80-91].

Despite being a leading cause of morbidity and mortality for transplant recipients, CAV surveillance strategies remain highly variable across centers, with little progress being made in tailoring surveillance strategies to individual patient risk profiles [Chih, et al, cited above; Loupy, A., et al., Identification and Characterization of Trajectories of Cardiac Allograft Vasculopathy After Heart Transplantation. Circulation, 2020. 141(24): p. 1954-1967; DePasquale, E.C., Predicting the Future of Cardiac Allograft Vasculopathy With Cardiac Positron Emission Tomography. Circulation: Heart Failure, 2018. 11(6): p. e005136]. While the usage of regular endomyocardial biopsy (EMB) remains the universal standard for acute rejection monitoring, for CAV monitoring, some centers opt for less invasive approaches with annual stress testing, with others opting for a more invasive approach with annual or periodic coronary angiography.

Regardless of the specific surveillance approach employed, all current methods for CAV detection rely on repurposing techniques initially developed for traditional coronary artery disease. As such, these techniques focus primarily on estimating the flow of blood through large, epicardial coronary arteries. Despite the macroscopic focus of commonly used CAV diagnostics, the pathobiology of CAV involves not only narrowing of the large muscular arteries of the heart, but also significant (and often antecedent) micro- vascular inflammation and injury to the capillaries and smaller pre-capillary arterioles [Brenevel et al, cited above; Chih, et al, cited above; Eisen, H.J., et al., Everolimus for the prevention of allograft rejection and vasculopathy in cardiac-transplant recipients. N Engl J Med, 2003. 349(9): p. 847-58; Hiemann Nicola, E., et al., Prognostic Impact of Microvasculopathy on Survival After Heart Transplantation. Circulation, 2007. 116(11): p. 1274-1282]. The importance of microvascular injury to end-organ function has been demonstrated in pathology studies of the native heart [Kaze, A.D., et al., Microvascular Disease and Incident Heart Failure Among Individuals With Type 2 Diabetes Mellitus. Journal of the American Heart Association, 2021. 10(12): p. e018998; Rush, C.J., et al., Prevalence of Coronary Artery Disease and Coronary Microvascular Dysfunction in Patients With Heart Failure With Preserved Ejection Fraction. JAMA Cardiology, 2021; Climie, R.E., et al., Macrovasculature and Microvasculature at the Crossroads Between Type 2 Diabetes Mellitus and Hypertension. Hypertension, 2019. 73(6): p. 1138-1149[, the transplanted heart Hiemann Nicola, E., et al., Prognostic Impact of Microvasculopathy on Survival After Heart Transplantation. Circulation, 2007. 116(11): p. 1274-1282; Revelo, M.P., et al., Longitudinal evaluation of microvessel density in survivors vs. nonsurvivors of cardiac pathologic antibody-mediated rejection. Cardiovasc Pathol, 2012. 21(6): p. 445-54; Loupy, A., et al., Late Failing Heart Allografts: Pathology of Cardiac Allograft Vasculopathy and Association With Antibody-Mediated Rejection. Am J Transplant, 2016. 16(1): p. 111-20; van den Hoogen, P., et al., Cardiac allograft vasculopathy: a donor or recipient induced pathology? Journal of cardiovascular translational research, 2015. 8(2): p. 106-116], and in other solid organ transplants [Gupta, A., et al., Clinical and molecular significance of microvascular inflammation in transplant kidney biopsies. Kidney International, 2016. 89(1): p. 217-225; Doreille, A., M. Dieudé, and H. Cardinal, The determinants, biomarkers, and consequences of microvascular injury in kidney transplant recipients. American Journal of Physiology-Renal Physiology, 2018. 316(1): p. F9-F19; Ishii, Y., et al., Injury and progressive loss of peritubular capillaries in the development of chronic allograft nephropathy. Kidney International, 2005. 67(1): p. 321-332]. Moreover, pathologic studies have consistently demonstrated that histologically apparent changes to the microarchitecture of vessels and tissue precede and predict eventual macroscopic findings and overt graft dysfunction [Loupy, 2016, cited above; Ishii, et al, cited above; Tsuji, T., et al., Microvascular inflammation in early protocol biopsies of renal allografts in cases of chronic active antibody-mediated rejection. Nephrology (Carlton), 2015. 20 Suppl 2: p. 26-30]. Yet despite several decades of published literature describing histopathologic changes consistent with microvascular disease in CAV, no standardized, objective framework for measuring these changes has been developed.

Multiple studies have attempted to identify clinical metrics that might be predictive of future CAV [Loupy et al, 2020, cited above; Mehra, M.R., et al., International Society for Heart and Lung Transplantation working formulation of a standardized nomenclature for cardiac allograft vasculopathy; 2010. The Journal of Heart and Lung Transplantation, 2010. 29(7): p. 717-727; López-Sainz, Á., et al., Late graft failure in heart transplant recipients: incidence, risk factors and clinical outcomes. European Journal of Heart Failure, 2018. 20(2): p. 385-394; Seki, A. and M.C. Fishbein, Predicting the development of cardiac allograft vasculopathy. Cardiovasc Pathol, 2014. 23(5): p. 253-60; Mehra Mandeep, R., et al., Predictive model to assess risk for cardiac allograft vasculopathy: An intravascular ultrasound study. Journal of the American College of Cardiology, 1995. 26(6): p. 1537-1544; Watanabe, K., S.D. Miyamoto, and S.J. Nakano, Predicting Cardiac Allograft Vasculopathy Using Circulating Vascular Endothelial Growth Factor in Pediatric Heart Transplant Recipients. The Journal of Heart and Lung Transplantation, 2017. 36(4): p. S78-S79; Sato, T., et al., Risk Stratification for Cardiac Allograft Vasculopathy in Heart Transplant Recipients ― Annual Intravascular Ultrasound Evaluation -. Circulation Journal, 2016. 80(2): p. 395-403]. The most successful of these efforts have attempted to incorporate not only baseline donor and recipient characteristics, but also several post-transplant metrics describing a predisposition to allo-immunity (including a history of acute cellular rejection (ACR) or donor specific antibodies (DSA) in the first year).

However, the performance on an individual patient level of these studies remains uncertain [Moayedi, Y. and J.J. Teuteberg, Predicting Where Patients Will Be, Rather Than Just Seeing Where They Are. Circulation, 2020. 141(24): p. 1968-1970], and as a result, the clinical impact of these efforts has been limited. To date, no study has attempted to pursue a rigorous analysis of the histologic data contained within routine EMB tissues as a means of generating better, more personalized CAV risk assessments. This has been considered can be considered unmet need [Alfonso, F., F. Rivero, and J. Segovia-Cubero, Early diagnosis of cardiac allograft vasculopathy: biopsy, liquid biopsy, non-invasive imaging, coronary imaging, or coronary physiology? European Heart Journal, 2021] and an emerging opportunity [Duong Van Huyen, J.P., et al., The XVth Banff Conference on Allograft Pathology The Banff Workshop Heart Report: Improving the Diagnostic Yield from Endomyocardial Biopsies and Quilty Effect Revisited. Am J Transplant, 2020], given the promise computational digital pathology analysis has shown for providing clinically valuable predictions in heart transplant medicine [Nirschl, J.J., et al., A deep-learning classifier identifies patients with clinical heart failure using whole-slide images of H&E tissue. PLOS ONE, 2018. 13(4): p. e0192726; Peyster, E.G., et al., An automated computational image analysis pipeline for histological grading of cardiac allograft rejection. European Heart Journal, 2021. 42(24): p. 2356-2369; Peyster, E.G., et al., In Situ Immune Profiling of Heart Transplant Biopsies Improves Diagnostic Accuracy and Rejection Risk Stratification. JACC: Basic to Translational Science, 2020. 5(4): p. 328-340] and beyond [Janowczyk, A., et al., High-Throughput Biomarker Segmentation on Ovarian Cancer Tissue Microarrays via Hierarchical Normalized Cuts. IEEE Transactions on Biomedical Engineering, 2012. 59(5): p. 1240-1252; Leo, P., et al. Combination of nuclear NF-kB/p65 localization and gland morphological features from surgical specimens is predictive of early biochemical recurrence in prostate cancer patients. in SPIE Medical Imaging. 2018. SPIE; Lu, C., et al., An oral cavity squamous cell carcinoma quantitative histomorphometric-based image classifier of nuclear morphology can risk stratify patients for disease-specific survival. Mod Pathol, 2017. 30(12): p. 1655-1665; Bulten, W., et al., Automated deep-learning system for Gleason grading of prostate cancer using biopsies: a diagnostic study. Lancet Oncol, 2020. 21(2): p. 233-241; Ström, P., et al., Artificial intelligence for diagnosis and grading of prostate cancer in biopsies: a population-based, diagnostic study. Lancet Oncol, 2020. 21(2): p. 222-232].

### SUMMARY OF THE INVENTION

Provided herein are computer-implemented computational image analysis of digitized EMB histology slides using an interpretable, methodology which identified and measures novel histologic biomarkers associated with the development of CAV. These biomarkers, along with relevant clinical data, were useful in the design of a computer-implemented integrated clinical and histologic model for predicting the development of early, severe CAV and for improved, personalized, methods of treatment.

In certain embodiments, a method for diagnosing, predicting and/or preventing acute allograft vasculopathy in an organ transplant patient is provided. The method comprises executing on a processor the steps of: (a) analyzing clinical risk factors of a cardiac, kidney or liver transplant patient in a model which distinguishes between clinical biomarkers of allograft vasculopathy patients and non-allograft vasculopathy patients, and (b) analyzing digital biopsy images from the transplant patient to morphologically differentiate patients which are prE allograft vasculopathy or which have active allograft vasculopathy from patients without acute allograft vasculopathy; (c) assigning prE-, active or non- allograft vasculopathy status to a patient based on the outcome of (a) and (b); and (d) where preE or active status is assigned, treating the patient. The digital biopsy images comprise at least one stain specific for endothelial cells and/or connective tissues of the vascular cells and tissues. In certain embodiments, the stain CD31, CD34, CD68, or combinations thereof. In other embodiments, the stain is a connective tissue stain, optionally selected from Mason’s trichrome or Movats pentachrome stain. In certain embodiments, the analysis of morphologic biomarkers in (b) and (c) comprises analysis of (i) interstitial composition and proliferation, (ii) myocyte density, (iii) total microvascular density, (iv) the proportions of vessels of different sizes, and (v) the cellular abundance both within microvessels and in the immediate perivascular space around these vessels.

In certain embodiments, the patient is a cardiac transplant patient and the digital biopsy images are from an endomyocardial biopsy (EMB) obtained at about 1 year post-transplant. In certain embodiments, the patient is assigned prE or active cardiac allograft vasculopathy status.

In certain embodiments, the patient is assigned positive status for pre-early vasculopathy or active vasculopathy if certain combinations of pathology findings are present: changes in overall stromal content area in a connective tissue stained slide; increased interstitial collagen content; increased non-collagen interstitial stromal content; increased thickness of staining of individual vascular structures; increased cellularity within stained vascular structures; increased cellularity in the perivascular area immediately surrounding stained vascular structures; and/or decreased overall tissue-density of vascular structures.

In certain embodiments, the method is used for diagnosing active cardiac allograft vasculopathy. In such embodiments, the analyzing step (b) comprises analyzing: percentage of total vascular nuclei found within larger capillaries and small pre-capillary arterioles; number Nuclei within vasculature as normalized by myocyte area; number of Nuclei outside the vascular/perivascular space as normalized by myocyte area; percentage of total nuclei in perivascular space as normalized by myocyte area; number of Nuclei within in vasculature as normalized by stromal area; number of Nuclei outside the vasculature/perivascular space as normalized by stromal area; Total stroma to myocardium ratio; Collagen content of interstitium; percentage of vascular nuclei around medium microvessels as normalized by stromal area, and Total vascular density as determined by vascular object area to myocardium area; and Interstitial stromal area.

In certain embodiments, the method is used for predicting acute allograft vasculopathy and step (b) comprises analyzing: Interstitial stroma as normalized by myocardial compartment size; percentage of microvessels that are larger capillaries and small pre-capillary arterioles; Non-collagen/non-proteoglycan content of interstitium; number of perivascular nuclei per vessel area for pre-capillary arterioles; total stroma to myocardium ratio; percentage of total DAB/vascular area comprised of pre-capillary arterioles; total microvascular staining area as normalized by myocardium; microvascular staining area of pre-capillary arterioles as normalized; total # of nuclei in and around vessels as normalized by myocardium; and percentage of total vascular nuclei found within capillaries.

In certain embodiments, the method comprises analyzing the clinical risk factors comprising: (i) actively treated recipient diabetes at one-year post-transplant, (ii) recipient body mass index at one-year post transplant, (iii) recipient low-density lipoprotein at one year post-transplant, (iv) a history of high-grade cellular rejection or treated rejection in first year, (v) the percentage of biopsies in the first year with Quilty lesion, (vi) donor proteinuria, and/or (vi) donor coronary angiography score.

In certain embodiments, the method comprises analyzing (a) History of Cellular Rejection, Recipient BMI, Donor Coronary Angiography Score, and Recipient Diabetes and (b) Interstitial stroma as normalized by myocardial compartment size; Non-collagen/non-proteoglycan content of interstitium; number of perivascular nuclei per vessel area for pre-capillary arterioles; Total stroma to myocardium ratio; Total microvascular staining area as normalized by myocardium; Microvascular staining area of pre-capillary arterioles as normalized; Total number of nuclei in and around vessels as normalized by myocard, and percentage of total vascular nuclei found within capillaries.

In certain embodiments, a patient assigned a pre-allograft vasculopathy or active allograft vasculopathy assigned (a positive diagnosis) is treated with a regimen comprising one or more immunosuppressants, one or more of a statin, mycophenolate mofetil, everolimus, sirolimus, aspirin, vitamins, a PCSK-9 inhibitor, a P2y12 inhibitor, ezetimibe, and/or fish oil.

In certain embodiments, wherein the patient is a heart transplant patient. In certain embodiments, the patient is a kidney transplant patient.

In certain embodiments, a computer-implemented method for detection of a pre-CAV or active CAV patient is provided which comprises executing on a processor the steps of: (a) analyzing clinical risk factors of a transplant patient (e.g., cardiac or kidney) in a model which distinguishes between clinical biomarkers of (i) prE-allograft vasculopathy (e.g., CAV) or active allograft vasculopathy (e.g., CAV) patient and (ii) non-prE-allograft vasculopathy or non-allograft vasculopathy patients, and (b) analyzing digital biopsy (e.g., EMB) images from the transplant patient to morphologically differentiate (i) prE-allograft vasculopathy (e.g., CAV) or active allograft vasculopathy (e.g., CAV) patient and (ii) non-prE-allograft vasculopathy or non-allograft vasculopathy patients; and (c) assigning (i) PrE-allograft vasculopathy (e.g., CAV) or active allograft vasculopathy (e.g., CAV) or (ii) non-PrE-allograft vasculopathy (e.g., non-prE-CAV) or non-allograft vasculopathy (e.g., non-CAV) status to a patient.

In certain embodiments, a non-transitory computer-readable storage medium comprising stored instructions is provided which, when executed by one or more computer processors, cause the one or more computer processors to perform the steps comprising: (a) analyzing clinical risk factors of a cardiac transplant patient in a model which distinguishes between clinical biomarkers of (i) prE-CAV or active CAV and (ii) non-prE-CAV or non-CAV patients, and (b) analyzing digital endomyocardial biopsy (EMB) images from the cardiac transplant patient to morphologically differentiate (i) PrE-CA V or active CAV from (ii) non-prE-CAV patients; and (c) assigning PrE-CAV or non-PrE-CAV status to a patient.

In certain embodiments, a system for detection of a pre-CAV or active CAV patient, comprising at least one processor configured to perform steps comprising: (a) analyzing clinical risk factors of a cardiac transplant patient in a model which distinguishes between clinical biomarkers of (i) prE-CAV or active CAV patients and (ii) non-prE-CAV or non-CAV patients, and (b) analyzing digital endomyocardial biopsy (EMB) images from the cardiac transplant patient to morphologically differentiate (i) prE-CAV or active CAV patients and (ii) non-prE-CAV or non-CAV patients; said at least one processor being configured to PrE-CAV or non-PrE-CAV status to a patient.

### BRIEF DESCRIPTION OF THE DRAWINGS

Various features of the embodiments described in the following detailed description can be more fully appreciated when considered with reference to the accompanying figures, wherein the same numbers refer to the same elements.

FIG. 1 provides a flow diagram for cardiac allograft vasculopathy (CAV) experiments. N=746 transplant recipients from the University of Pennsylvania between 2007-2020 underwent detailed health record review to collect baseline data at time of transplant, donor data, 1-year-post-transplant clinical data, and long-term CAV outcomes data. Patients were labeled as ‘No-CAV’ if they did not have any evidence of CAV by six-years-post-transplant, and were labeled as ‘Pre-Early-CAV’ (PrE-CAV) if they developed moderate or severe CAV by five-years post-transplant (but not before 1-year post-transplant). N=444 did not meet either definition, due to lack of CAV diagnosis (thus not meeting PrE-CAV definition), or due to <6 years of follow-up (thus unable to meet No-CAV definition). N=302 patients met a study group definition, and were included in study analyses. Based on clinical data available at 1-year post-transplant, a clinical risk factor model for predicting future CAV was developed to distinguish between No-CAV and PrE-CA V patients (the ClinCAV-Pr model). Additionally, n=183 archival biopsies from study patients were collected, stained, and digitized to undergo digital pathology image analysis for ‘morphologic biomarker’ extraction. ‘PrE-CAV biopsies’ and ‘No-CAV biopsies’ were comprised of low-grade, one-year post-transplant surveillance biopsy tissues, while ‘Disease-Control’ (DC) biopsies were biopsies obtained from patients at the time of definitive moderate/severe CAV diagnosis. Morphologic biomarker models were developed to distinguish between overt CAV biopsies and No-CAV biopsies (the HistoCAV-Dx model) and to predict future CAV development based on 1-year post-transplant No-CAV and PrE-CAV biopsies (the HistoCAV-Pr model). Finally, to test the added predictive value of novel morphologic biomarkers, clinical risk factors and morphologic biomarkers were combined in an integrated ‘iCAV-Pr’ model.

FIGS. 2A to 2F provide illustrative digital histology images for 1-year post-transplant biopsies from pre-Early CAV (PrE-CAV) patients prior to disease onset (FIG. 2C) and 1-year post-transplant (FIG. 2D) from non-early CAV (No-CAV) patients (FIG. 2A and FIG. 2B), as well as Disease Control (DC) biopsies obtained from CAV patients at the time of definitive disease diagnosis (FIGS. 2E and 2F). Note the progressive interstitial/stromal changes in Movat’s images for both DC (FIGS. 2E and 2F) and PrE-CA V patients (FIGS. 2C and 2D), with prominent collagen (arrows)) expansion in the DC EMBs. Also note the perivascular cellular density in PrE-CAV and DC biopsies, as well as notable increases in thickness of the DAB (brown) stain in the CD31 DC image suggesting endothelial cell hypertrophy or proliferation. These and other, subtler features were extracted from these images during image analysis, and used to develop the experimental Histologic Diagnostic and Predictive models for CAV (histoCAV-Dx and HistoCAV-Pr, respectively).

FIG. 3 provides an image analysis workflow for cardiac allograft vasculopathy diagnosis and prediction from endomyocardial biopsy samples. The top row shows the workflow for CD31 stained slides, starting with a raw digital image and proceeding left to right with detection of CD31 stained vascular objects (dark brown in raw image, solid green after object detection and segmentation), color-unmixing and optical density algorithms to resolve nuclei (blue) underlying CD31 vascular objects, nuclear object detection and segmentation both in and around these vascular objects, and finally, differentiation of perivascular (blue) vs. intra-vascular (red) nuclei for deeper characterization of vascular morphology changes. Bottom row shows the workflow for the corresponding Movats stained slides. The first segmentation steps identify all stromal fibers (both mucopolysaccharides/glycosaminoglycans [GAGs] in darker green and collagen in lighter green) and all myocytes (red). This is followed by generation of a ‘myocardial mask’ which encompasses the tissue compartment containing significant myocyte density, and sub-analysis of the stromal fibers contained within this myocardial mask to quantify and contrast interstitial stromal changes with more global stromal findings.

FIG. 4A provide a standard deviation boundary curve and FIG. 4B illustrates a DAB object area (µm²).

FIGS. 5A and 5B show the performance in the study test set of a clinical risk factor model using six previously validated multivariate predictors of cardiac allograft vasculopathy (CAV) trajectories. The overall performance of this six variable model is similar to but worse than the performance of the Clinical CAV Prediction Model (ClinCAV-Pr) developed in this experiment, which included several novel risk factors related to baseline patient risk for vasculopathy and/or coronary artery disease.

FIG. 6 provides visual examples with explanatory descriptions of key morphologic biomarkers that comprise the Histologic Cardiac Allograft Vasculopathy Diagnostic (HistoCAV-Dx) and Predictive (HistoCAV-Pr) Models. Visual examples with explanatory descriptions of morphologic biomarker categories that comprise the Histologic Cardiac Allograft Vasculopathy Diagnostic (HistoCAV-Dx) and Predictive (HistoCAV-Pr) Models. 1st Row: increased interstitial collagen content (green arrows pointing to yellow staining between red myocytes) in a disease control (DC) tissue sample obtained at the time of definitive cardiac allograft vasculopathy (CAV) diagnosis (right image right) vs. a No-CAV tissue sample (left image panel), as seen in a Movats pentachrome stained slide. 2nd Row: Increased non-collagen interstitial stromal content (green arrows pointing to blue-grey staining between red myocytes) in a pre-early-CAV tissue sample (PrE-CAV) vs. a No-CAV sample (Movats pentachrome). Note that PrE-CAV and No-CAV samples are obtained at 1-year post-transplant, and that PrE-CAV samples are obtained before overt CAV onset. 3rd Row: Increased thickness of CD31 staining in vascular structures (green arrows pointing to brown areas), likely representing increased endothelial cell thickening/abundance in a DC sample vs. a No-CAV sample (CD31 stained slide). 4th Row: Increased cellularity (green arrows pointing to blue nuclei) within CD31 vascular structures (brown) in a DC sample vs. No-CAV (CD31 stained slide). 5th Row: Increased cellularity (green arrows pointing to blue nuclei) in the perivascular area immediately surrounding CD31 vascular structures (brown), as seen in a PrE-CA V sample vs. a No-CAV sample (CD31 stained slide). 6th Row: Area of decreased overall ‘vascular density’, with sparser brown-staining CD31 vascular structures in a DC sample vs. a No-CAV sample (CD31 stained slide).

FIGS. 7A and 7B illustrate HistoCAV-Dx, a model for diagnosing cardiac allograft rejection (CAV) using automated image analysis of endomyocardial biopsy (EMB) tissues, was trained on n=88 EMBs. Excellent diagnostic performance was achieved in an independent test set of n=45 EMBs, with area under the receiver operating characteristic curve and sensitivity both in excess of 90% for differentiating tissue from patients with CAV vs. those without CAV.

FIGS. 8A ― 10B provide the results for three experimental models designed to predict early/aggressive cardiac allograft vasculopathy (CAV) before overt disease onset using only data available at one-year post-transplant.

FIGS. 8A and 8B provide the performance of the Clinical risk factor Prediction Model (ClinCAV-Pr) for predicting CAV in an independent test set (n=95 patients) after model training using clinical data from n=207 patients. Overall, this seven-variable model relying on clinical risk factors achieves modest predictive performance, though with a poor true positive rate and a true negative rate that largely benefits from an imbalanced cohort with a relatively low frequency of CAV.

FIGS. 9A and 9B provide the Performance of a Histologic Prediction Model (HistoCAV-Pr) for predicting CAV, based on automated histologic analysis of one-year post-transplant endomyocardial biopsies (EMB). After model training on n=88 EMBs, the model performance was assessed on an independent test set of n=44 EMBs corresponding to cases present in the ClinCAV-Pr test-set described above. Overall, good performance was achieved by the HistoCAV-Pr, with clear improvements in area under the ROC curve, accuracy, sensitivity, and positive predictive value compared to ClinCAV-Pr.

FIGS. 10A and 10B provide the performance of an Integrated ‘histo-clinical’ CAV prediction model (iCAV-Pr) for predicting CAV, which combines the prediction outputs of ClinCAV-Pr and HistoCAV-Pr. There is clear synergy when combining broad clinical risk factors and computationally extracted morphologic biomarkers, with excellent predictive performance that exceeds either model alone.

### DETAILED DESCRIPTION OF THE INVENTION

The systems and methods provided herein illustrate the utility of transplant biopsy sample analysis for diagnosing and predicting allograft vasculopathy in heart transplant, kidney transplant, and lung transplant tissues. In certain embodiments, the systems and methods are used for predicting, diagnosing and/or treatment of in heart transplant patients using, e.g., cardiac biopsy (e.g., endomyocardial biopsy or EMB) which comprises myocytes and other cardiac vascular and connective tissues. In other embodiments, the systems and methods are used for predicting, diagnosing and/or treatment of allograft vasculopathy in lung transplant patients using, e.g., biopsies comprising pulmonary tissue comprising vascular and connective tissues and cells. In other embodiments, the systems and methods are used for predicting, diagnosing and/or treatment of renal allograft vasculopathy in kidney transplant patients using, e.g., renal vascular and connective tissue and cells.

The quantitative histologic analysis method deployed in this work permits not only the characterization of morphologic biomarkers associated with advanced allograft rejection (e.g., CAV), but demonstrated that certain biomarkers are present in vascular biopsy (e.g., EMB) samples years before overt CAV development. These predictive morphologic biomarkers were not only superior to traditional clinical risk factors, but were also orthogonal to them, enabling superior CAV risk assessments when morphologic predictors and clinical predictors are combined. In the examples herein, morphologic biomarkers describing the proliferation of interstitial fibers and the ‘cellularity’ of the intra- and peri-vascular space demonstrate the strongest predictive potential for future vasculopathy development.

In certain embodiments, the utilization of computer implemented methods within cardiovascular, nephrology, and/or pulmonary transplant medicine.

Without wishing to be bound by theory, important to the development of the methods and system provided herein, is the inventors’ finding that both morphologic models of image features describing the cellularity of the perivascular space highlights an area for deeper mech anistic study. Importantly, endothelitis and endothelial hyperplasia/proliferation are both early (occurring as soon as 1-year post transplant) and persistent finding (present at the time of conventional CAV diagnosis) findings. The presence of these factors has not previously described as being present at the early stage found by the inventors. In certain embodiments in which the patient has CAV, the use of cardiac microvascular morphologic biomarkers with preserved ejection fractions are combined with selected clinical biomarkers to predict diabetes or heart failure.

One advantage of the methods provided herein is finding that broad clinical risk factors, while also acknowledging the need to look beyond traditional risk factors via advanced morphologic analysis of routinely acquired EMB tissues.

In one embodiment, a method for diagnosing, predicting and/or preventing acute allograft vasculopathy in an organ transplant patient. The method involves executing a series of steps on a processor, including analyzing clinical risk factors of a cardiac, kidney or liver transplant patient in a model which distinguishes between clinical biomarkers of allograft vasculopathy patients and non-allograft vasculopathy patients, analyzing digital biopsy images from the transplant patient to morphologically differentiate patients which are prE allograft vasculopathy or which have active allograft vasculopathy from patients without acute allograft vasculopathy; and assigning prE-, active or non- allograft vasculopathy status to a patient based on the outcome of (a) and (b).

For many transplant patients, post-transplant biopsies (i.e., collection of tissues) is part of the routine standard of care. For example, for cardiac transplant patients, endomyocardial biopsies (EMB) are routinely performed at about 1-year (or about 330 days to about 400 days post-transplant). For kidney transplant patients, renal biopsies may be performed at about six (6) months to about twelve (12) months. For lung transplant patients, biopsies (e.g., transbronchial tissue) may be performed at about two months to about nine months post-lung transplantation. Thus, the methods provided herein may be readily integrated into the current standard of care. However, the methods provided may be used on tissue samples (biopsies) obtained at different time points in order to predict allograft vasculopathy and/or to diagnose suspected acute allograft vasculopathy. In certain embodiments, the sample is obtained a patient about two months to 10 years post-transplant.

While the collection of tissue samples to monitor patients post-transplant is part of the current standard of care, the types of staining described herein for preparing digitized images of the biopsy samples used in the methods provided herein is not routine. Prior to preparing digitized images of the biopsy samples, the samples are stained to facilitate detection of endothelial cells and connective tissues and components thereof, e.g., the interstitium, microvessels, stroma, collagen, non-collagen interstitial tissue, non-proteoglycan content, vascular nuclei, perivascular nuclei, sizes of vessels, and other cellular and tissue components described herein.

In certain embodiments, once the biopsy sample (e.g., EMB for CAV diagnosis or prediction) is obtained, the tissues are prepared for staining with cluster differential 31 (CD31) according to conventional staining methods for other cell types.. CD31 also known as platelet endothelial cell adhesion molecule or PECAM-1). Suitable instructions may be obtained from the manufacturer of the stain, e.g., ThermoFischer. Additionally or alternatively, other endothelial cell satins may be used, e.g., CD34, CD31, CD68, or combinations thereof. Still other stains may be selected.

A separate subset of the biopsy sample may be prepared by staining with stains selective for connective tissue. For example, cells from the sample can also prepared for staining with Movat’s pentachrome [“Pathology News: Newsletter, Vol. 3, No. 4: April 1996” . Department of Pathology and Molecular Medicine, Queen’s University; Movat, HZ (1955). “Demonstration of all connective tissue elements in a single section; pentachrome stains”. AMA Archives of Pathology. 60 (3): 289-95. PMID 13248341; Russell Jr, HK (1972). “A modification of Movat’s pentachrome stain”. Archives of Pathology. 94 (2): 187-91. PMID 4114784.; and med.upenn.edu/merc/histology_core/movat.shtml, Modified Movat’s Pentachrome Stain has been described. Other suitable stains for connective tissues may include, e.g., trichrome stain, van Gieson’s stains, picrosirum red stain, Masson’s trichrome, among others. Such stains may be purchased commercially, e.g., from AbCAM. These stains are designed for use in electron microscopes

In certain embodiments, a quantitative histology pipeline for CAV may utilizes only hematoxylin and eosin (H&E) stained slides may be selected for use in the method, in the place of the selectively stained (e.g., CD31) endothelial cell stained slides, or the selectively stained (e.g., Movats pentachrome) connective tissue slides, or both.

The various embodiments provided herein can identify early microvascular manifestations of allograft vasculopathy (e.g., CAV) with high sensitivity and specificity. In certain embodiments, the methods combine digital pathology ‘morphologic biomarkers’ with clinical risk factors to identify patients who will develop aggressive cardiac, kidney or lung allograft vasculopathy years before overt disease onset. This permits allograft vasculopathy (e.g., CAV, kidney, or lung) prediction and tailoring both screening and preventative therapeutic strategies to individual allograft vasculopathy (e.g., CAV, kidney or liver) risk profiles.

Suitably, digital images are generated which comprise the endothelial cell stained cells (e.g., CD31) and the connective tissue stained cells (e.g., Movats pentachrome) for use in a machine-implemented methods provided herein.

### Morphologic Biomarkers of Acute Allograft Vasculopathy

The digitized images from an EMB (cardiac transplant patient), lung biopsy, or renal biopsy is analyzed for morphologic biomarkers of allograft vasculopathy. This may involve analysis of two, three, four, or all five of the following biomarkers from the digitized image of the stained biopsy sample: (i) interstitial composition and proliferation, (ii) myocyte density, (iii) total microvascular density, (iv) the proportions of vessels of different sizes, and (v) the cellular abundance both within microvessels and in the immediate perivascular space around these vessels. In certain embodiments, the patient is assigned positive status for pre-acute vasculopathy or active vasculopathy if the biomarkers is present: increased overall stromal content area in a connective tissue stained slide; increased interstitial collagen content; increased non-collagen interstitial stromal content; increased thickness of staining of individual vascular structures; increased cellularity within stained vascular structures; increased cellularity in the perivascular area immediately surrounding stained vascular structures; and/or decreased overall tissue-density of vascular structures.

In certain embodiments, the table below illustrates the morphologic biomarkers used to diagnose active allograft vasculopathy.

| | |
|---|---|
| Variable | Variable Description |
| prop_dab_nuc_in_q23 | % of total vascular nuclei found within larger capillaries and small pre-capillary arterioles |
| nuc_in_dab_by_myocard | # Nuclei within vasculature, normalized by myocyte area |
| nuc_outside_by_myocard | # Nuclei outside the vascular/perivascular space, normalized by myocyte area |
| nuc_dens_in_dilonly_by_m yo_dens_ | % of total nuclei in perivascular space, normalized by myocyte area |
| nuc_in_dab_by_stroma_in_ myocard | # Nuclei within in vasculature, normalized by stromal area |
| nuc_outside_by_stroma_in_ myocard | # Nuclei outside the vasculature/perivascular space, normalized by stromal area |
| total_stroma_by_myocard | Total stroma to myocardium ratio |
| collagen_inmyocard_by_myocard | Collagen content of interstitium |
| compstat 1 | % of vascular nuclei around medium microvessels, normalized by stromal area |
| dab_area_by_perc_myo_in_ myocard | Total vascular density (vascular object area to myocardium area) |
| stro_myocar_original | Interstitial stromal area |

In certain embodiments, diagnosing active cardiac allograft vasculopathy (CAV), comprises analyzing percentage of total vascular nuclei found within larger capillaries and small pre-capillary arterioles; number Nuclei within vasculature as normalized by myocyte area; number of Nuclei outside the vascular/perivascular space as normalized by myocyte area; percentage of total nuclei in perivascular space as normalized by myocyte area; number of Nuclei within in vasculature as normalized by stromal area; number of Nuclei outside the vasculature/perivascular space as normalized by stromal area; Total stroma to myocardium ratio; Collagen content of interstitium; percentage of vascular nuclei around medium microvessels as normalized by stromal area, and Total vascular density as determined by vascular object area to myocardium area; and Interstitial stromal area.

As used herein, the term “capillaries” refers to vessels which are generally smaller than about 25 µm² to about 65 µm². When reference it made to “larger capillaries and small pre-capillary arterioles”, the size range is generally from about 25 µm² to about 95 µm². When reference is made to “medium to large microvessels”, the size range is from about 65 µm to about 500 µm². Generally, this means that about 10% or less of the microvessels in the view, preferably about 5% or less of the microvessels are outside the recited size range.

The following table provides an illustrative binning parameters for the vascular sizes.

Table: Biologically inspired boundary definitions/Biological Bin Definitions/Boundaries

| | |
|---|---|
| Capillaries | < 78.4 µm²(< 10 µm in minor axis luminal diameter) |
| Small Precapillary Arterioles | 78.5 µm² to 314 µm² |
| Large Precapillary Arterioles | 315 µm² to 1000 µm² |
| Arterioles | > 1000 µm² |
| Large Outliers | 99.9% cutoff |

In certain embodiment, the biomarkers used predicting allograft vasculopathy comprise those set forth in the following table.

Table: Variables in HistoCAV-Pr for predicting future cardiac allograft vasculopathy, with annotated descriptions.

| | |
|---|---|
| Variable | Variable Description |
| stroma_in_myocard_by_myocard | Interstitial stroma, normalized by myocardial compartment size |
| prop_dab_obj_in_dab_s234 | % of microvessels that are larger capillaries and small pre-capillary arterioles |
| white_in_myocard_by_myocard | Non-collagen/non-proteoglycan content of interstitium |
| nuc_in_q34_dil_by_area_in_q34 | # perivascular nuclei per vessel area for pre-capillary arterioles |
| total_stroma_by_myocard | Total stroma to myocardium ratio |
| prop_of_dab_area_in_dab_s56_ | % of total DAB/vascular area comprised of pre-capillary arterioles |
| dab_area_by_myocard | Total microvascular staining area, normalized by myocardium |
| dab_s56_area_by_myocard | Microvascular staining area of pre-capillary arterioles, normalized |
| nuc_in_dildab_by_myocard | Total # of nuclei in and around vessels, normalized by myocard |
| prop_dab_nuc_in_q2 | % of total vascular nuclei found within capillaries |

In certain embodiments, predicting CAV comprises analyzing biomarkers comprising: Interstitial stroma as normalized by myocardial compartment size; percentage of microvessels that are larger capillaries and small pre-capillary arterioles; Non-collagen/non-proteoglycan content of interstitium; number of perivascular nuclei per vessel area for pre-capillary arterioles; total stroma to myocardium ratio; percentage of total DAB/vascular area comprised of pre-capillary arterioles; total microvascular staining area as normalized by myocardium; microvascular staining area of pre-capillary arterioles as normalized; total # of nuclei in and around vessels as normalized by myocardium; and percentage of total vascular nuclei found within capillaries. Parallel biomarkers are selected for predicting renal allograft vasculopathy, although the sample is obtained from renal and perinephretic tissue rather than cardiomyoctes. In another embodiment, parallel embodiments are selected for lung transplant patients using bronchial tissue samples. In certain embodiments, prE-allograft vasculopathy patient (e.g., preE-CAV) is assigned wherein the computer-implemented analysis of the digital biopsy identifies one or more of: increased overall stromal content area in a Movats pentachrome stained slide; increased interstitial collagen content; increased non-collagen interstitial stromal content; increased CD31 staining of vascular structures; increased cellularity within CD31 vascular structures; increased cellularity in the perivascular area immediately surrounding CD31 vascular structures; and/or area of decreased overall vascular density.

### Analysis Clinical Biomarkers

The systems and methods provided herein combine analysis of cellular morphology biomarkers described above with clinical risk factors. For cardiac transplant patients, the clinical risk factors analyzed are two, three, four, five, six, of the following risk factors, combinations thereof, or all seven risk factors. The clinical risk factors comprise: (i) actively treated recipient diabetes at one-year post-transplant, (ii) recipient body mass index at one-year post transplant, (iii) recipient low-density lipoprotein at one year post-transplant, (iv) a history of high-grade cellular rejection or treated rejection in first year, (v) the percentage of biopsies in the first year with Quilty lesion, (vi) donor proteinuria, and/or (vi) donor coronary angiography score.

For pre-CAV or an active CAV diagnosis, the method may comprise analyzing:clinical biomarkers: History of Cellular Rejection, Recipient BMI, Donor Coronary Angiography Score, and Recipient Diabetes and analyzing morphologic biomarkers: Interstitial stroma as normalized by myocardial compartment size; Non-collagen/non-proteoglycan content of interstitium; number of perivascular nuclei per vessel area for pre-capillary arterioles; Total stroma to myocardium ratio; Total microvascular staining area as normalized by myocardium; Microvascular staining area of pre-capillary arterioles as normalized; Total number of nuclei in and around vessels as normalized by myocard, and percentage of total vascular nuclei found within capillaries.

In certain embodiments, the method and systems provided herein analyze the following clinical and morphologic biomarkers:

| | |
|---|---|
| t | Variable Description |
| stroma_in_myocard_by_myocard | Interstitial stroma, normalized by myocardial compartment size |
| white_in_myocard_by_myocard | Non-collagen/non-proteoglycan content of interstitium |
| nuc_in_q34_dil_by_area_in_q34 | # perivascular nuclei per vessel area for pre-capillary arterioles |
| total_stroma_by_myocard | Total stroma to myocardium ratio |
| dab area by_myocard | Total microvascular staining area, normalized by myocardium |
| dab_s56_area_by_myocard | Microvascular staining area of pre-capillary arterioles, normalized |
| nuc_in_dildab_by_myocard | Total # of nuclei in and around vessels, normalized by myocard |
| prop_dab_nuc_in_q2 | % of total vascular nuclei found within capillaries |
| History of Cellular Rejection | ISHLT grade 2R or 3R in first year post-transplant |
| Recipient BMI | At 1-year post-transplant |
| Donor Coronary Angiography Score | Derived from the Heart Donor Score, and incorporating donor age as a consideration |
| Recipient Diabetes | With active treatment at 1-year post-transplant |

ISHLT = international society for heart and lung transplantation, BMI = body mass index, LDL= low density lipoprotein

Methods for performing the various scoring and grading are known to those of skill in art. Normalization of the various values may be performed as described in the Examples section herein or by using other techniques available to those of skill in the art. For example, the ISHLT grading and coronary angiography scores are described in the literature. See, e.g., Stewart S, Winters GL, Fishbein MC, Tazelaar HD, Kobashigawa J, Abrams J, Andersen CB, Angelini A, Berry GJ, Burke MM, Demetris AJ, Hammond E, Itescu S, Marboe CC, McManus B, Reed EF, Reinsmoen NL, Rodriguez ER, Rose AG, Rose M, Suciu-Focia N, Zeevi A and Billingham ME. Revision of the 1990 working formulation for the standardization of nomenclature in the diagnosis of heart rejection. J Heart Lung Transplant. 2005;24:1710-20; and Smits JM, De Pauw M, de Vries E, Rahmel A, Meiser B, Laufer G and Zuckermann A. Donor scoring system for heart transplantation and the impact on patient survival. J Heart Lung Transplant. 2012;31:387-97.

In still a further embodiment, the computer-implemented system analyzes patient clinical biomarkers and the results of the morphologic analysis in order to assign a patient a prE-CAV status or a non-preE-CAV status. The clinical biomarkers includes (1) % prior biopsies with Quilty lesions [also known as endocardial infiltrates; Billingham ME. Cardiac Transplantation. In: Sale GE, ed. The Pathology of Organ Transplantation. Boston: Butterworths, 1990:133-152.] (2) average grade of preceding biopsies; (3) number of high-grade biopsies; and (4) history of any treated rejection, and may also include more convention clinical data, e.g., lab results, diagnoses, demographics. These may be readily represented by a continuous number scale (e.g. Ages, weights, blood counts etc.) or a binary scale (e.g. 0/1 or ‘yes’/‘no’) and statistical modeling is applied to these data.

| | Standardized Cardiac Biopsy Grading |
|---|---|
| Grade | Histopathological Findings |
| 0 | No rejection |
| 1 (1A or 1B) | A = Focal (perivascular or interstitial) infiltrate without necrosis |
| | B = Diffuse but sparse infiltrate without necrosis |
| 2 | One focus only with aggressive infiltration and/or focal myocyte damage |
| 3 (3A or 3B) | A = Multifocal aggressive infiltrates and/or myocyte damage |
| | B = Diffuse inflammatory process with necrosis |
| 4 | Diffuse aggressive polymorphous ± infiltrate ± edema, ± hemorrhage, ± vasculitis, with necrosis |
| Additional Required Information^{∗} | |
| Biopsy less than 4 pieces | |
| Humoral rejection (positive IF, vasculitis, or severe edema in absence of cellular infiltrate | |
| “Quilty” effect Type A or Type B) | A = No myocyte encroachment |
| | B = With myocyte encroachment |
| Ischemia | A = Up to 3 weeks posttransplant |
| | B = Late ischemia |

Infection present - biopsy therefore uninterpretable
Lymphoproliferative disorder
Other (specify)
^{∗}Must be added to biopsy report if present

### Treatment Patients Assigned prE-allograft or Active Allograft Vasculopathy Status

Using the methods and systems provided herein, a transplant patient may be assigned a status corresponding to (i) non active allograft vasculopathy or (ii) active allograft vasculopathy (positive). Alternatively, a transplant patient may be assigned a status corresponding to (i) non pre-active allograft vasculopathy (negative) or (ii) active pre-allograft vasculopathy (positive). In cardiac transplant patients, the allograft vasculopathy is CAV.

Where a patient is assigned a status of active allograft vasculopathy (e.g., CAV) or pre-allograft vasculopathy, the patient may be treated with a suitable therapeutic or prophylactic regimen. Such a regimen may include one or more of: one or more immunosuppressants, one or more of a statin, mycophenolate mofetil, everolimus, sirolimus, aspirin, vitamins, a PCSK-9 inhibitor, a P2y12 inhibitor, ezetimibe, and/or fish oil.

The assignment of a patient with a non-pre-allograft vasculopathy or non-active allograft vasculopathy indicates that no therapeutic intervention is required, thus, avoiding unnecessarily treatment and potentially reducing the number/frequency of diagnostic tests for these heart transplant patients.

In certain embodiments, a computer-implemented method for detection of a pre-CAV or active CAV patient is provided which comprises executing on a processor the steps comprising: (a) analyzing clinical risk factors of a cardiac transplant patient in a model which distinguishes between clinical biomarkers of (i) prE-CAV or active CAV patient and (ii) non-prE-CAV or non-CAV patients, and (b) analyzing digital endomyocardial biopsy (EMB) images from the cardiac transplant patient to morphologically differentiate (i) PrE-CA V or active CAV from (ii) non-prE-CAV or non-CAV patients; and (c) assigning (i) PrE-CAV or active CAV or (ii) non-PrE-CAV or non-CAV status to a patient.

In certain embodiments, a computer-implemented method for detection of a pre-renal allograft vasculopathy (RAV) or active renal allograft vasculopathy (RAV) patient is provided which comprises executing on a processor the steps comprising: (a) analyzing clinical risk factors of a renal transplant patient in a model which distinguishes between clinical biomarkers of (i) prE-RAV or active RAV patient and (ii) non-prE-RAV or non-RAV patients, and (b) analyzing digital renal biopsy images from the cardiac transplant patient to morphologically differentiate (i) PrE-RAV or active RAV from (ii) non-prE-RAV or non-RAV patients; and (c) assigning (i) PrE-RAV or active RAV or (ii) non-PrE-RAV or non-RAV status to a patient.

In certain embodiments, a non-transitory computer-readable storage medium comprising stored instructions which, when executed by one or more computer processors, cause the one or more computer processors to perform steps comprising: (a) analyzing clinical risk factors of a cardiac transplant patient in a model which distinguishes between clinical biomarkers of (i) prE-CAV or active CAV and (ii) non-prE-CAV or non-CAV patients, and (b) analyzing digital endomyocardial biopsy (EMB) images from the cardiac transplant patient to morphologically differentiate (i) PrE-CA V or active CAV from (ii) non-prE-CAV patients; and (c) assigning PrE-CAV or non-PrE-CAV status to a patient.

In certain embodiments, a non-transitory computer-readable storage medium comprising stored instructions which, when executed by one or more computer processors, cause the one or more computer processors to perform steps comprising: (a) analyzing clinical risk factors of a renal transplant patient in a model which distinguishes between clinical biomarkers of (i) prE-RAV or active RAV patient and (ii) non-prE-RAV or non-RAV patients, and (b) analyzing digital renal biopsy images from the cardiac transplant patient to morphologically differentiate (i) PrE-RAV or active RAV from (ii) non-prE-RAV or non-RAV patients; and (c) assigning (i) PrE-RAV or active RAV or (ii) non-PrE-RAV or non-RAV status to a patient.

In certain embodiments, a system for detection of a pre-CAV patient is provided. The system comprises at least one processor configured to perform steps comprising: (a) analyzing clinical risk factors of a cardiac transplant patient in a model which distinguishes between clinical biomarkers of (i) prE-CAV or active CAV patients and (ii) non-prE-CAV or non-CAV patients, and (b) analyzing digital endomyocardial biopsy (EMB) images from the cardiac transplant patient to morphologically differentiate (i) prE-CAV or active CAV patients and (ii) non-prE-CAV or non-CAV patients; said at least one processor being configured to PrE-CAV or non-PrE-CAV status to a patient.

In certain embodiments, a system for detection of a pre-CAV patient is provided. The system comprises at least one processor configured to perform steps comprising: (a) analyzing clinical risk factors of a renal transplant patient in a model which distinguishes between clinical biomarkers of (i) prE-RAV or active RAV patient and (ii) non-prE-RAV or non-RAV patients, and (b) analyzing digital renal biopsy images from the cardiac transplant patient to morphologically differentiate (i) PrE-RAV or active RAV from (ii) non-prE-RAV or non-RAV patients; and (c) assigning (i) PrE-RAV or active RAV or (ii) non-PrE-RAV or non-RAV status to a patient.

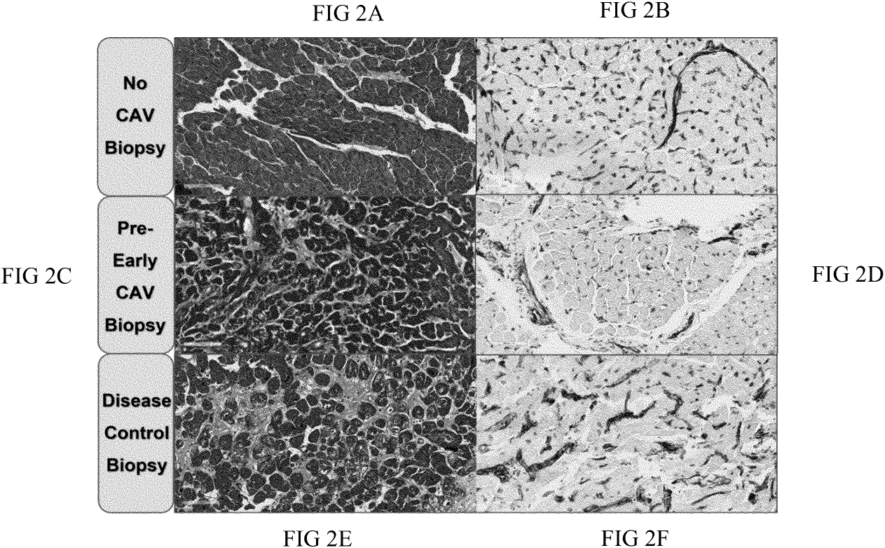

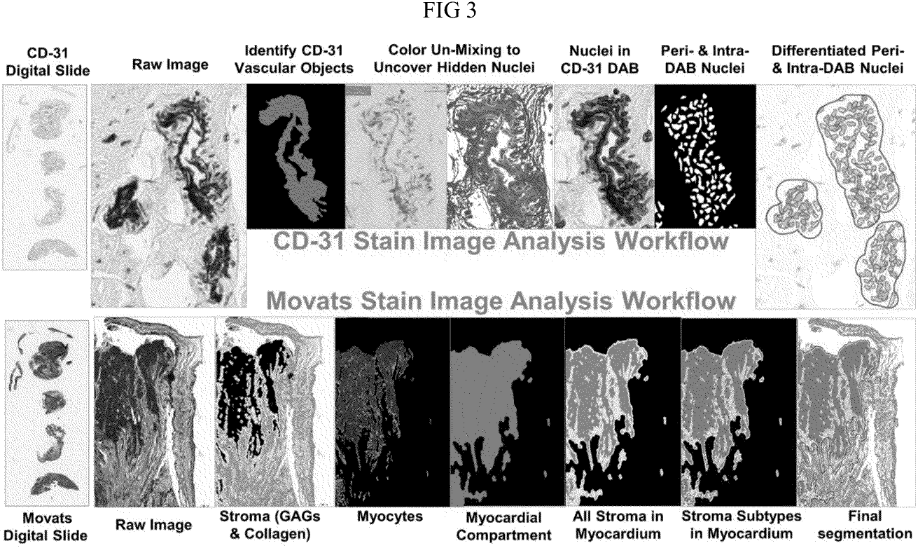

In certain embodiments, the computer-implemented system performs a series of automated nuclei and vascular object detection on the digitized image of the CD31-stained cells from the EMB sample. These analytic methods peri- and intra-vessel nuclei detection and differentiation. See, e.g., FIG. 2, which illustrates a workflow comprising identifying CD-31-stained vascular objects, color un-mixing to uncover hidden nuclei, nuclei in CD-31 DAB, peri and intra-nuclei DAB, and differentiated peri and intra-DAB nucleic. Variation in the order of this analysis is permitted.

In certain embodiments, the computer-implemented system further comprises automated segmentation and/or automated quantitation of myocytes, collagen and other fibers. The first segmentation steps identify all stromal fibers (both mucopolysaccharides/glycosaminoglycans [GAGs]. This is followed by generation of a ‘myocardial mask’ which encompasses the tissue compartment containing significant myocyte density, and sub-analysis of the stromal fibers contained within this myocardial mask to quantify and contrast interstitial stromal changes with more global stromal findings. For example, the following from a digitized Movats stained EMB sample may be assessed for one or more of: Stroma (glycosaminoglycans (GAGs) and collagen), myocytes, myocardial compartment, all stroma in myocardium, stroma subtypes in myocardium, and final segmentation.

In the examples below, the parameters used for running positive cell detection using the CD31 stained cells were as follows, including Setup parameters (detection image: hematoxylin OD image requested pixel size (0.5 µm)), Nucleus Parameters: ((background radius: 8 µm, median filter radius: 0 µm, sigma: 1.5 µm, minimum area: 10 µm²; maximum area: 400 µm²); Intensity Parameters: (threshold 0.1; maximum background intensity: 2); Split by Shape; Cell parameters; Cell expansion (0 µm, include cell nucleus); General parameters: Smooth boundaries and Make measurements; Intensity threshold parameters: ((Score compartment, nucleus: DAB OD mean); Threshold 1: 0.2, Threshold 2 (0.4), Threshold 3 (0.6), single threshold). Variations on these parameters are permitted and will be apparent to one of skill in art both when using CD31-stained cells and when selecting different visualization (stains) in view of the present specification.

It is to be noted that the term “a” or “an” refers to one or more. As such, the terms “a” (or “an”), “one or more,” and “at least one” are used interchangeably herein.

The words “comprise”, “comprises”, and “comprising” are to be interpreted inclusively rather than exclusively. The words “consist”, “consisting”, and its variants, are to be interpreted exclusively, rather than inclusively. While various embodiments in the specification are presented using “comprising” language, under other circumstances, a related embodiment is also intended to be interpreted and described using “consisting of” or “consisting essentially of” language.

As used herein, the term “about” means a variability of 10% from the reference given, unless otherwise specified.

A “patient” is a preferably a human, although the methods may be tested in a suitable model, e.g., mouse, rat, guinea pig, dog, cat, horse, cow, pig, or non-human primate, such as a monkey, chimpanzee, baboon or gorilla. In one embodiment, the patient is a human.

Unless defined otherwise in this specification, technical and scientific terms used herein have the same meaning as commonly understood by one of ordinary skill in the art and by reference to published texts, which provide one skilled in the art with a general guide to many of the terms used in the present application.

### EXAMPLE

A specific example of use of the above disclosed method is provided below. This method is not a limitation on the invention.

In this proof-of-concept study, we perform computational image analysis of digitized EMB histology slides using an interpretable, computer-implemented methodology in order to discover and measure novel histologic biomarkers associated with the development of CAV. Further, we utilize these biomarkers, along with relevant clinical data, to develop an integrated clinical and histologic model for predicting the development of early, severe CAV.

Cardiac allograft vasculopathy (CAV) is a leading cause of morbidity and mortality for heart transplant recipients. While clinical risk factors for CAV have been established, no personalized prognostic test exists to confidently identify patients at high vs. low risk of developing aggressive CAV. The aim of this investigation was to leverage computational methods for analyzing digital pathology images from routine endomyocardial biopsies (EMB) to develop a precision medicine tool for predicting CAV years before overt clinical presentation.

Methods: Clinical data from 1-year post-transplant was collected on 302 transplant recipients from the University of Pennsylvania, including 53 ‘early CAV’ patients and 249 healthy controls. This data was used to generate a ‘clinical model’ for predicting future CAV development. From this cohort, n=183 archived EMBs were collected for CD31 and modified trichrome staining and then digitally scanned. These included 1-year post-transplant EMBs from 50 ‘early CAV’ patients and 82 no-CAV patients, as well as 51 EMBs from ‘disease control’ patients obtained at the time of definitive coronary angiography con- firming CAV. Using biologically-inspired, hand-crafted features extracted from digitized EMBs, quantitative histologic models for differentiating no-CAV from disease controls, and for predicting future CAV from 1-year post-transplant EMBs were developed. The performance of histologic (i.e. HistoCAV-Pr) and clinical (i.e., ClinCAV-Pr) models for predicting future CAV were compared in a held-out validation set, before being combined as to assess the added predictive value of an integrated predictive model (iCAV-Pr).

Results: ClinCAV-Pr achieved modest performance on the independent test set, with area under the receiver operating curve (AU- ROC) of 0.70. The histologic model for diagnosing CAV (i.e., HistoCAV-Dx) achieved excellent discrimination, with an AU- ROC of 0.91, while HistoCAV-Pr achieved good performance with an AUROC of 0.80. The integrated iCAV-Pr model achieved excellent predictive performance, with an AUROC of 0.93 on the held-out test set.

Conclusions: Prediction of future CAV development is greatly improved by incorporation of computationally extracted histologic features. These results suggest morphologic details contained within regularly obtained biopsy tissue have the potential to enhance precision and personalization of treatment plans for post-heart transplant patients.

Study Cohort and Design: The study cohort consisted of patient records and archived clinical histology samples from the University of Pennsylvania. Detailed clinical records up to the one-year anniversary of transplant were obtained from n=302 transplant recipients transplanted between 2004 and 2017. This included n=53 Pre-‘Early-CAV’ (PrE-CAV) patients, defined as patients who will go on to have an angiographic diagnosis of CAV with an International Society of Heart and Lung Transplantation (ISHLT) grade of 2 or 3 (denoting angiographic evidence of focal severe stenosis or diffuse disease with evidence of allograft dys-function)¹⁹ occurring within five years of transplantation. The remaining n=249 ‘No-CAV’ patients were defined as having no history of positive stress testing or angiographic evidence of CAV at six-years post-transplant, and were selected serially from patients transplanted between 2006 and 2014. Table 1A provides the clinical characteristics of the full study cohort.

For the development of an automated histology analysis pipeline, n=50 EMB tissue blocks obtained during one-year post- transplant visits from PrE-CAV patients, and n=82 EMB tissue blocks obtained during one-year post-transplant visits from No-CAV patients were retrieved from the pathology archives at the University of Pennsylvania. In addition, n=51 EMB tissue blocks from biopsy procedures performed at the time of definitive coronary angiography diagnosing patients with grade 2 or 3 CAV were also obtained to represent ‘disease control’ (DC) histology samples (these patients were not necessarily PrE-CAV patients, but instead are defined by the availability of EMB tissue at the time of definitive CAV diagnosis). Table 1B provides relevant biopsy sample characteristics for the cases undergoing automated histologic analysis.

TABLE 1A:

Clinical, histologic, and donor characteristics of early cardiac allograft vasculopathy patients, as compared to patients without allograft vasculopathy:

| | No-CAV (n=249) | PrE-CAV (n=53) | p Value (univariate) |
|---|---|---|---|
| RECIPIENTS: | | | |
| Age at OHT (yrs) | 52.6 | 48.9 | 0.020 |
| Sex (% Female) | 23.6 | 21.2 | 0.604 |
| White Ethnicity (%) | 83.6 | 78.8 | 0.452 |
| BMI at 1yr post OHT | 28.0 | 29.8 | 0.033 |
| Medical History: | | | |
| Hypertension (%) | 80.3 | 82.7 | 0.699 |
| Hyperlipidemia (%) | 82.2 | 86.5 | 0.472 |
| Diabetes Mellitus (%) | 57.5 | 75.0 | 0.042 |
| Coronary Artery Disease | 39.4 | 40.4 | 0.665 |
| CMV IgG Positive (%) | 42.9 | 50.9 | 0.343 |
| • CMV infection in 1st year | 6.0 | 15.1 | 0.024 |
| Histology History: | | | |
| Average ISHLT Grade of prior EMBs at 1 yr* | 0.71 | 0.78 | 0.163 |
| % of Prior EMBs with Quilty at 1 yr* | 13.2 | 23.0 | <0.001 |
| Treated Acute Rejection† in 1^{st} yr (%) | 44.3 | 50.0 | 0.002 |
| History of Positive PRA/CPRA (% of patients) | 20.8 | 13.2 | 0.200 |
| Positive Donor Specific Antibody in 1^{st} yr (%) | 11.2 | 11.3 | 0.987 |
| LDL Cholesterol at 1 yr post OHT (mg/dL) | 85.4 | 103.4 | <0.001 |
| Average LVEF at 1 year post OHT (%) | 65.0 | 64.1 | 0.881 |
| Age (yrs) | 39.1 | 40.0 | 0.676 |
| Sex (% Female) | 37.8 | 35.6 | 0.754 |
| White Ethnicity (%) | 75.6 | 73.1 | 0.624 |
| Smoking History (%) | 22.8 | 33.9 | 0.097 |
| BMI | 28.0 | 27.7 | 0.875 |
| Hypertension (%) | 22.8 | 22.6 | 0.919 |
| Diabetes (%) | 8.2 | 7.6 | 0.610 |
| Coronary Angiography Score‡ | 2.2 | 2.8 | 0.093 |
| Median Heart Donor Score36 | 16.8 | 17.1 | 0.801 |
| Organ Ischemic Time (avg, minutes) | 190.5 | 185.6 | 0.849 |
| Proteinuria at Harvest (%) | 42.1 | 56 | 0.072 |
| LVEF at Harvest (%) | 60.8 | 61.0 | 0.922 |
| Average number of HLA Mismatches | 4.5 | 4.7 | 0.849 |
| CMV Mismatch [Donor(-)/Recipient(+)] | 28.8 | 28.9 | 0.808 |
| Sex Mismatch (male donor to female recipient) | 21.6 | 23.1 | 0.879 |
| Race Mismatch | 35.2 | 45.3 | 0.079 |
| Serum BUN: Creatinine Ratio (average) | 14.8 | 13.8 | 0.340 |

*These are per-patient averages.
†Treated acute rejection refers to any treated rejection event, regardless of grade or sub-type.
‡ Coronary Angiography Score is derived from the validated EuroTransplant Heart Donor Score³⁶, and assumes the lowest score for donors 34 years of age and younger if no angiography is performed at harvest.
PrE-CAV = pre-early cardiac allograft vasculopathy (CAV) patients, defined as patients experiencing overt CAV by 5-years post-trans- plant but without overt diagnosis at 1-year post-transplant. No-CAV = patients without overt CAV at 6-years post-transplant. OHT = orthotopic heart transplant, ISHLT = international society for heart and lung transplantation, LVEF= Left ventricular ejection fraction, BMI = body mass index, LDL= low density lipoprotein, CMV= cytomegalovirus.

TABLE 1B

Histologic characteristics of the endomyocardial biopsy cases used for automated histologic analysis

| | No-CAV (n=82) | PrE-CAV (n=50) | Disease Controls (n=51) |
|---|---|---|---|
| Days post-transplant biopsy obtained (average) | 365 | 358 | 2350 |
| ISHLT Grade 0R (n) | 41% (34) | 24% (12) | 35.3% (18) |
| ISHLT Grade 1R (n) | 57.8% (48) | 76% (38) | 64.7% (33) |
| ISHLT Grade 2R/3R (n) | 0% | 0% | 0% |
| Quilty Lesion (n) | 6% (5) | 30% (15) | 15.7% (8) |
| Prior Biopsy Changes (n) | 6% (5) | 6% (3) | 3.9% (2) |
| C4d+ on Immunofluorescent staining^{§} (n) | 0% | 0% | 19.6% (10) |
| Immunopathologic Antibody Mediated Rejection / pAMR1-i+ (n) | 0% | 0% | 3.9% (2) |
| Digitized on Hamatsu slide scanner (vs. Aperio) | 60% (49) | 60% (30) | 54.9% (28) |

ISHLT = International Society for Heart and Lung Transplantation.
§C4d+ on IF staining describes any positive staining, even if insufficient for a diagnosis of antibody mediated rejection/pAMR-i+. pAMR1-i+ = ISHLT grade 1 with immunofluorescence sufficiently positive to be consistent with antibody mediated rejection (AMR).

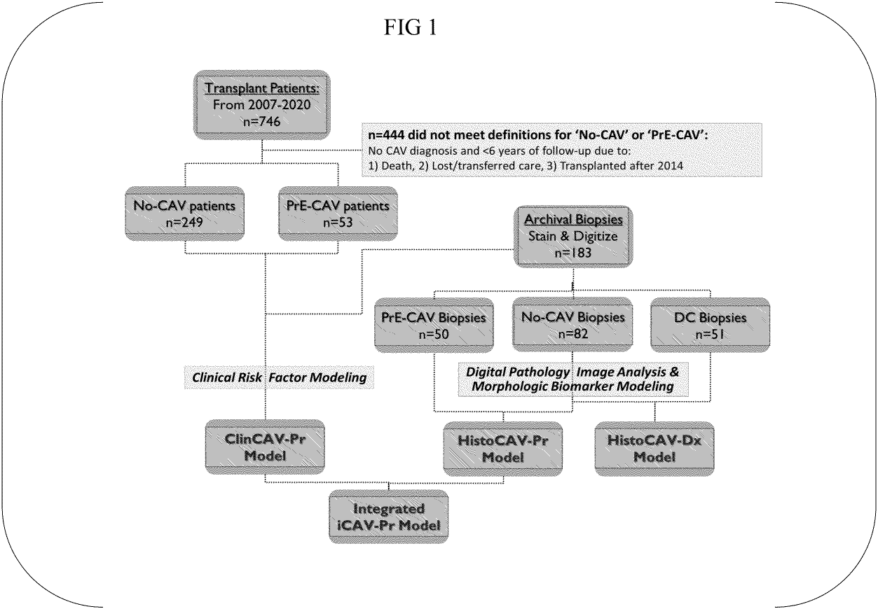

Histology Slide Preparation: All tissue samples were fixed in 4% paraformaldehyde and embedded in paraffin at the time of EMB procedure, as per routine care. Archival residual EMB tissue blocks were retrieved from pathology archives for this research, with two new serial 10 micron sections cut from each block for each study case. One section was stained with Movat’s pentachrome (a modified trichrome stain permitting discrimination of ground substance/mucopolysaccharides from collagen and reticular fibers), while the other underwent immunohistochemistry with CD31 (Ab28364, Abcam, Cambridge, UK, with 3,3′-Diaminobenzidine labeling) and hematoxylin to highlight endothelial cells. Digital histology images were generated via whole-slide scanning at 40× magnification using either an Aperio ScanScope or a Hamatsu S360 slide scanner. Two different slide scanners were utilized to enable assessment of image analysis pipeline performance on different scanner devices. FIGS. 1A-F shows digital histology images for No-CAV, PrE-CAV, and DC EMBs.

Image Quality Control: Image quality control is an essential component of digital image analysis research. Digitized slides underwent quality control assessments using HistoQC, an open-source, automated digital pathology analysis software tool for identifying arti-facts and measuring slide quality which we have successfully applied to previous investigations in cardiac histology [Peyster, E.G., et al., An automated computational image analysis pipeline for histological grading of cardiac allograft rejection. European Heart Journal, 2021. 42(24): p. 2356-2369; Janowczyk, A., et al., HistoQC: An Open-Source Quality Control Tool for Digital Pathology Slides. JCO Clinical Cancer Informatics, 2019(3): p. 1-7].

Overview of Image Analysis Workflow: The image analysis workflow focused on extracting relevant morphologic features from the CD31 and Movat’s stained images separately, prior to combining the feature sets to generate quantitative variables during data analysis. Using observational information relating to interstitial expansion/remodeling in the setting of subacute/chronic allograft inflammation, endothelitis/endothelial cell thickening in CAV, and cellular proliferation and migration in the vessel wall or perivascular space during indolent allo-immunity as inspiration, we attempted to extract measurable features that capture these phenomena. Analysis was conducted on whole-slide EMB images, with specific workflows summarized below and in FIG. 2.

CD31 Workflow: The primary purpose of the CD31 slides was to facilitate and enhance the accuracy of morphologic biomarkers describing the microvasculature. The open-source digital pathology tool QuPath (version 0.2.3) was employed for raw image data extraction from CD31 slides, with a focus on identifying 3,3′-Diaminobenzidine (DAB)-stained endothelial cells and hematoxylin-stained nuclei. Stain deconvolution algorithms enabled reliable separation of areas containing specific ― and even overlapping ― stain-colors [Ruifrok, A. and D. Johnston, Ruifrok AC, Johnston DA. Quantification of histochemical staining by color deconvolution. Anal Quant Cytol Histol 23: 291-299. Analytical and quantitative cytology and histology / the International Academy of Cytology [and] American Society of Cytology, 2001. 23: p. 291-9]. Following stain deconvolution, three pixel-level classifiers were created for assigning pixels to (a) the CD31/DAB class, (b) the hematoxylin class, (c) remaining tissue class. Python (using the Shapely and Pandas libraries) was used for quantitative feature development via the measurement of areas, counts, densities, and specific arrangements of classified pixels. To ensure a comprehensive and nuanced evaluation of the cardiac microvasculature, and due to uncertainty over which type/size of microvessel might provide the most valuable predictive information, several ‘binning’ strategies for sub-classifying vascular (e.g. DAB-staining) objects by size were explored. This involved first removing the extreme outliers of size (DAB+ objects with areas in the top 0.5% of the entire cohort), which represent either rare and sporadically distributed large vascular objects at the border between micro- and macro-vessels with diameters >~120 µm [Climie, R.E., et al., Macrovasculature and Microvasculature at the Crossroads Between Type 2 Diabetes Mellitus and Hypertension. Hypertension, 2019. 73(6): p. 1138-1149]. Next, DAB regions were binned using three different approaches: (a) statistical criteria (i.e., 0.5, 1, 1.5, 2, 3, and >3 standard deviations away from the mean), (b) evenly-spaced quartiles, and (c) previously defined anatomical criteria [Hiemann Nicola, E., et al., Prognostic Impact of Microvasculopathy on Survival After Heart Transplantation. Circulation, 2007. 116(11): p. 1274-1282], as the Table with Biologically inspired boundary definitions.

More particularly, the CD31 Workflow utilized QuPath-0.2.3, an open source software platform for digital pathology and WSI image analysis, was used for data extraction from CD31 slides. HistoQC software was downloaded from github using this repository, https://github.com/choosehappy/HistoQC.

Segmentation: Annotations and Nuclei Detection: To initiate feature discrimination, the WSIs stained with CD31 were imported into a new QuPath project. The following steps were completed using Apache Groovy scripts: (a) Preprocessing; (b): Annotation; (c) Nuclei detection.

Preprocessing: Preprocessing included setting the image type (Brightfield H-DAB), background, and stain deconconvolution. These were the parameters used:

setColorDeconvolutionStains(‘{“Name” : “H-DAB CD31”,
“Stain 1” : “Hematoxylin”, “Values 1” : “0.59666 0.75249 0.27885”,
“Stain 2” : “DAB”, “Values 2” : “0.24042 0.48171 0.84271”,
“Background” : “ 235 235 243 ”}’);

Annotation: The following pixel classifiers and their corresponding parameters were generated in order to annotate specific regions or features of interest within the annotated ROI. Resolution parameters may differ based on the slides.
a. A Region of Interest (ROI) was annotated using a pixel classifier and classified as “CD31ROI”. The following threshold values were used: Resolution: Low (4.02 µm/px), Channel: Average, Prefilter: Gaussian, Smoothing Sigma: 4, Threshold: 231, Minimum object size: 4000 µm², and Minimum hole size: 4000 µm².
b. CD31Tissue_classifier_v3: Resolution: Very High (0.50 µm/px), Channel: Average, Prefilter: Gaussian, Smoothing Sigma: 4, Threshold: 235, Minimum object size: 10 µm², and Minimum Hole Size: 10 µm².
c. CD31DAB_classifier_v3: Resolution: Full (0.25 µm/px), Channel: DAB, Prefilter: Gaussian, Smoothing Sigma: 1.5, Threshold: 0.2, Minimum object size: 10 µm², and Minimum Hole Size: 10 µm²
d. CD31HEMA_classifier_v3: Resolution: Full(0.25 µm/px), Channel: Hematoxylin, Prefilter: Gaussian, Smoothing Sigma: 1.5, Threshold: 0.2, Minimum object size: 10 µm², and Minimum Hole Size: 10 µm²

Nuclei Detection: Having classified the annotations, positive cell detection was run with the following parameters on the “CD31 Tissue” annotations. FIG. 3 provides the parameters used for running positive cell detection. The annotation and nuclei data was exported as GeoJSON files for each WSI. Each WSI file therefore has a corresponding nuclei.json file and annotation.json file. The corresponding nuclei and annotation GeoJSON files were then opened using Python’s Geojson and Shapely libraries. Areas for tissue, DAB, hematoxylin, and nuclei were calculated using the Shapely library and collected in four respective Pandas dataframes. The four data frames were then saved to tis.csv, dab.csv, hem.csv, and nuc.csv. Each of these files contain the cohort, filename, and area. In dab.csv, the corresponding Shapely polygons and polygon coordinates were also saved. Next, in order to assess the perivascular environment, detection of nuclei within a small area around DAB regions was performed, which involved slightly dilating DAB regions (proportionally to their size) and counting cells falling within this expansion. A full set of quantitative measurements were generated for the total DAB+ object population across the whole slide and for each size bin from each binning approach. Counts were aggregated at the DAB bin level for usage as features in model creation.

Extracting Features: Binning and DAB dilation: Dab.csv was loaded in a pandas dataframe and rows where DAB areas were in the bottom 0.01% were dropped to compensate for errors.

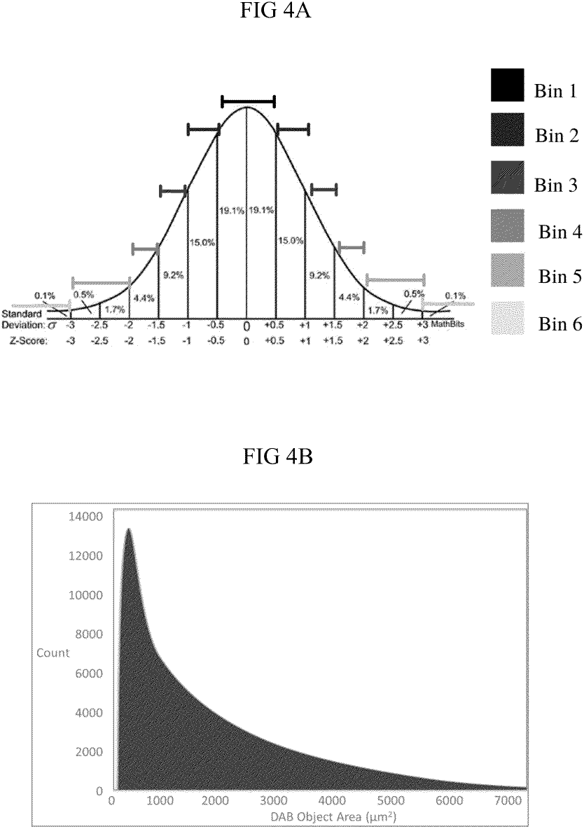

Defining Boundaries for Various Binning Processes
1. Quartile bin boundaries were calculated using statistics from the csv dataframe for the 25th, 50th, and 75th percentiles.
2. Standard deviation bin boundaries, outlined in Supplemental Figure S.2, were calculated by the addition of a new column to the data frame that holds the z-score for that row. The z-score was calculated using the equation:
$$\frac{A r e a - M e a n}{S t a n d a r d D e v i a t i o n} .$$
The boundaries were then defined as a) within ±0.5 standard deviations from the mean b) within ±1.0 standard deviation excluding the area between ±0.5 standard deviations c) within ±1.5 standard deviation excluding the area between ±1.0 standard deviations d) within ±2.0 standard deviation excluding the area between ±1.5 standard deviations e) within ±3.0 standard deviation excluding the area between ±2.0 standard deviations f) outside of ±3.0 standard deviations. See, FIG. 4.

Biological bin boundaries were defined as follows (conversion used: area of 1 pixel² = 0.0633 µm²):

Table: Biologically inspired boundary definitions/Biological Bin Definitions/Boundaries

| | |
|---|---|
| Capillaries | < 78.4 µm² (< 10 µm in minor axis luminal diameter) |
| Small Precapillary Arterioles | 78.5 µm² to 314 µm² |
| Large Precapillary Arterioles | 315 µm² to 1000 µm² |
| Arterioles | >1000 µm² |
| Large Outliers | 99.9% cutoff |

Dab areas in the top 0.1 % were classified as outliers and treated as a separate bin.

DAB Dilation and Binning Process: The DAB dataframe was grouped by cohorts and filename. For each group, its corresponding nuc.json file was opened and its nuclei detections were read using shapely and geojson. Shapely’s unary_union was used for dilating the DAB annotation, STRtree was used for searching nuclei centroids, and Shapely’s Within function was used to identify nuclei within DAB. Nuclei that were within overlapping dilated regions were not double counted. The following process was performed by dilating the DAB annotation based on the bins. The Table below outlines the bin dilation sizes.
findNucsInDAB_qrt: identify nuclei that were within quartile boundaries
findNucsInDAB_std: identify nuclei that were within standard deviation boundaries
findNucsInDAB_bio: identify nuclei that were within biological boundaries
findNucsInDAB_excl: identify nuclei that were within the excluded DAB boundaries

Table: Binning strategies and their corresponding bin dilation sizes.

| Quartile Bins | | Standard Deviation Bins | | Biological Bins | |
|---|---|---|---|---|---|
| Bin | Dilation (pixels) | Bin | Dilation (pixels) | Bin | Dilation (pixel) |
| q1 | 2 px | s1 | 2 px | b1 | 2 px |
| q2 | 5 px | s2 | 5 px | b2 | 5 px |
| q3 | 10 px | s3 | 10 px | b3 | 10 px |
| q4 | 15 px | s4 | 15 px | b4 | 15 px |
| | | s5 | 20 px | b5 | 20 px |
| | | s6 | 25 px | | |

The final CD31 results were collected and saved to CSV.

Movats Workflow: The primary purpose of Movats staining was to achieve reliable quantitation of myocytes and various stromal fiber types within EMB tissue samples. Movat’s Pentachrome stained slides include differentially stained regions of myocytes, collagen, basement membrane, and elastin. Given the highly orthogonal stain colors, after preprocessing, thresholds in the HSV color space were employed to create individual binary masks for each stain color/fiber type. After modest post-processing (e.g., hole filling and dilation), Python’s NumPY library was used to calculate the total area for each of the binary masks per WSI, before being stored for subsequent interrogation. Recognizing that dense areas of stroma on the endocardial surface or in areas of prior biopsy site may have different implications from interstitial stromal proliferation, we also performed sub-analysis of stromal fiber content within the myocardial compartment alone (e.g., the areas of tissue which contain significant myocyte density). This was achieved via disc-dilation of the myocyte segmentation results, which created a confluent mask encompassing the myocyte-containing regions of tissue. Using this ‘myocardium mask’, the different stromal fibers could be measured to assess their relative contributions to interstitial changes in the tissue.

Movat’s Pentachrome classification started with extracting 2000 × 2000 tiles from the WSI.

Segmentation: Movat’s Pentachrome stained tiles included regions of myocytes, collagen, basement membrane, stroma, and nuclei. The following Table presents each of these features, their corresponding color, and the threshold range used.

| | | |
|---|---|---|
| Feature | Color | Threshold Range |
| Myocyte | Red | L_RED _MIN = np.array([0,20,70]) |
| | | L_RED_MAX = np.array([5,255,255]) |
| | | U_RED _MIN = np.array([140,20,70]) |
| | | U_RED_MAX = np.array([180,255,255]) |
| Collagen | Yellow | YELLOW_MIN = np.array([5, 20, 70],) |
| | | YELLOW_MAX = np.array([60, 255, 255]) |
| Basement Membrane | Blue | BLUE_MIN = np.array([60, 20, 70],) |
| | | BLUE_MAX = np.array([140, 255, 255]) |
| Stroma and Nuclei | Black | BLACK_MIN = np.array([1,1,1]) |
| | | BLACK_MAX = np.array([255,255,70]) |
| Background | White | WHITE_MIN = np.array([0, 0, 180]) |
| | | WHITE_MAX = np.array([255,60, 255]) |

Tiled images were opened in the BGR color space using OpenCV and a Gaussian blur with a 5 x 5 kernel was applied to this image. A low pass filter, gaussian kernel, was applied in order to reduce random noise in the tiled images. This Gaussian blurred image was then converted to HSV. Feature detection in the HSV space was chosen because BGR includes luminance which makes feature discrimination difficult. HSV abstracts hue while separating illumination making it better suited for feature detection. Each stained feature correlated with a specific range of colors in HSV space defined in the table above.

However, certain slides were overstained and this created issues with the segmentation process which would lead to false positives for stromal detections. To address this, overstained images were manually identified and brightened using Python’s OpenCV convertScaleAbs with an alpha (contrast) value of 1.5 and a beta (brightness) value of 0 prior to segmentation.

Binary masks of each color’s minimum and maximum range were created. Before calculating the area of the binary masks, post processing using Skimage’s morphology functions was needed to reduce noise. The following Table describes the various morphological parameters used. A myocardium mask was also generated by filling holes in the myocyte mask and dilating it until large sheets of myocytes were confluent.

Table: Parameters for morphological masks.

| | |
|---|---|
| Color (Feature) | Post Processing Values |
| Red (Myocyte) | binary_erosion (boolean_mask, disk(3)) |
| | remove_small_objects(boolean_mask, |
| | min_size=400) |
| | remove_small_holes(boolean_mask, 400) |
| Yellow (Collagen) | binary_dilation(boolean_mask, disk(3)) remove_small_objects(boolean_mask, min_size=100) remove_small_holes(boolean_mask, 400) |
| Blue (Basement Membrane) | binary_dilation(boolean_mask, disk(3)) remove_small_objects(boolean_mask, min_size=100) remove_small_holes(boolean_mask, 100) |
| Black (Stroma and Nuclei) | remove_small_objects(boolean_mask, min_size=100) remove_small_holes(boolean_mask, 5000) |
| White (Background) | binary_dilation(boolean_mask,(prev_mask), disk(3)) remove_small_objects(boolean_mask, min_size=100) remove_small_objects(boolean_mask, min_size=100) remove_small_holes(boolean_mask, 5000) |
| Myocardium | binary_dilation(boolean_mask,(prev_mask), disk(30)) remove_small_holes(boolean_mask, 5000) remove_small_objects(boolean_mask, min_size=5000) binary_erosion(boolean_mask, disk(20)) |

Feature Quantification: After morphology functions were applied, Python’s NumPY was used to calculate the total area for each binary mask, where each binary mask was represented by a specific feature. The features were summed by WSI file and grouped by cohort. This Movats data was then saved to a CSV file.

Merging CD31 and Movats Data: CD31 and Movats data was merged using Pandas based on corresponding filenames. From this combined dataset, the final set of n=680 features for morphologic model construction were generated. This involved using the extracted parameters as detailed above to calculate different cell/nuclei, tissue-type, and DAB object ratios to assess normalized densities and areas-covered in different parts of the digital slides. Specific examples of generated features can be found in the multivariate results in the tables below.

Table: Variables in HistoCAV-Dx Model for Diagnosing cardiac allograft vasculopathy, with annotated descriptions.

| Variable | Variable Description | Multivariate p Value | Odds Ratio |
|---|---|---|---|
| prop_dab_nuc_in_q23 | % of total vascular nuclei found within larger capillaries and small pre-capillary arterioles | 0.002 | 0.806664 |
| nuc_in_dab_by_myocard | # Nuclei within vasculature, normalized by myocyte area | 0.023 | 0.581875 |
| nuc_outside_by_myocard | # Nuclei outside the vascular/perivascular space, normalized by myocyte area | 0.02 | 1.67034 |
| nuc_dens_in_dilonly_by_ myo_dens_ | % of total nuclei in perivascular space, normalized by myocyte area | 0.007 | 0.883957 |
| nuc_in_dab_by_stroma_i n_myocard | # Nuclei within in vasculature, normalized by stromal area | 0.039 | 1.555839 |
| nuc_outside_by_stroma_i n_myocard | # Nuclei outside the vasculature/perivascular space, normalized by stromal area | 0.049 | 0.685748 |
| total_stroma_by_myocard | Total stroma to myocardium ratio | 0.033 | 0.474698 |
| collagen_inmyocard_by_ myocard | Collagen content of interstitium | 0.001 | 1.4787 |
| compstat 1 | % of vascular nuclei around medium microvessels, normalized by stromal area | 0.043 | 1.086368 |
| dab_area_by_perc_myo_in_myocard | Total vascular density (vascular object area to myocardium area) | 0.067 | 0.835601 |
| stro_myocar_original | Interstitial stromal area | 0.033 | 1.216634 |

Table: Variables in HistoCAV-Pr for predicting future cardiac allograft vasculopathy, with annotated descriptions.

| Variable | Variable Description | Multivariate p Value | Odds Ratio |
|---|---|---|---|
| stroma_in_myocard_by _myocard | Interstitial stroma, normalized by myocardial compartment size | 0.006 | 1.711429 |
| prop_dab_obj_in_dab_s 234 | % of microvessels that are larger capillaries and small pre-capillary arterioles | 0.002 | 0.839721 |
| white_in_myocard_by_ myocard | Non-collagen/non-proteoglycan content of interstitium | 0.047 | 0.860249 |
| nuc_in_q34_dil_by_are a_in_q34 | # perivascular nuclei per vessel area for pre-capillary arterioles | 0.004 | 1.204962 |
| total_stroma_by_myocard | Total stroma to myocardium ratio | 0.001 | 0.767723 |
| prop_of_dab_area_in_dab_s56_ | % of total DAB/vascular area comprised of pre-capillary arterioles | 0.087 | 1.226093 |
| dab_area_by_myocard | Total microvascular staining area, normalized by myocardium | 0.008 | 2.998339 |
| dab_s56_area_by_myocard | Microvascular staining area of pre-capillary arterioles, normalized | 0.008 | 1.562746 |
| nuc_in_dildab_by_myocard | Total # of nuclei in and around vessels, normalized by myocard | 0.016 | 0.405156 |
| prop_dab_nuc_in_q2 | % of total vascular nuclei found within capillaries | 0.021 | 0.876805 |

Table: Variables in an integrated ‘Histo-Clinical’ Model optimized through backwards elimination of variables from the final ClinCav-Pr and the final HistoCAV-Pr Model. Note that this multivariate model was not used as the final iCAV-Pr model due to inferior performance compared to a simple two-variable model using classification probabilities from ClinCAV-Pr and HistoCAV-Pr.

| Variable | Variable Description | Multivariate p Value | Odds Ratio |
|---|---|---|---|
| stroma_in_myocard_ by_myocard | Interstitial stroma, normalized by myocardial compartment size | 0.045 | 2.30084 |
| white_in_myocard_by_myocard | Non-collagen/non-proteoglycan content of interstitium | 0.094 | 0.7725427 |
| nuc_in_q34_dil_by_ area_in_q34 | # perivascular nuclei per vessel area for pre-capillary arterioles | 0.025 | 1.339025 |
| total_stroma_by_myocard | Total stroma to myocardium ratio | 0.025 | 0.6811603 |
| dab_area_by_myocard | Total microvascular staining area, normalized by myocardium | 0.049 | 3.071829 |
| dab_s56_area_by_myocard | Microvascular staining area of pre-capillary arterioles, normalized | 0.022 | 0.6394363 |
| nuc_in_dildab_by_myocard | Total # of nuclei in and around vessels, normalized by myocard | 0.044 | 0.2901358 |
| prop_dab_nuc_in_q2 | % of total vascular nuclei found within capillaries | 0.032 | 0.7702914 |
| History of Cellular Rejection | ISHLT grade 2R or 3R in first year post-transplant | 0.052 | 1.358948 |
| Recipient BMI | At 1-year post-transplant | 0.025 | 1.157767 |
| Donor Coronary Angiography Score | Derived from the Heart Donor Score, and incorporating donor age as a consideration | 0.015 | 1.119375 |
| Recipient Diabetes | With active treatment at 1-year post-transplant | 0.098 | 1.021882 |

ISHLT = international society for heart and lung transplantation, BMI = body mass index, LDL= nsity lipoprotein
ISHLT = international society for heart and lung transplantation, BMI = body mass index, LDL= low density lipoprotein

Data Analysis and Statistical Methods: Image Feature Selection and Morphologic Model Construction:

The features derived from the above image analysis workflow describe the sizes, areas, and counts of a variety of key morphologic properties. These features extracted from both the CD31 and Movats workflows are then merged to derive variables for predictive modeling through the calculation of different cell/tissue-type/object ratios and via different normalization methods (e.g., dividing by total tissue area or myocardium area). In total, 680 quantitative features character izing the morphology of EMB tissues were computed for each case. These variables describe interstitial composition and proliferation, myocyte density, total vascular density, the proportions of vessels of different sizes, and the cellular abundance both within vessels and in the immediate perivascular space (FIG. 6). These features describe interstitial composition and proliferation, myocyte density, vascular density and the proportions of vessels of different sizes, and cellularity both within vessel walls and in the immediate perivascular space. These features were subsequently used to produce two distinct classification models ― a ‘Diagnostic Model’ (HistoCAV-Dx) to differentiate No-CAV EMBs from DC EMBs (total cohort n=133), and a ‘Morphologic Predictive Model’ (HistoCAV-Pr) to differentiate No-CAV EMBs from PrE-CAV EMBs (total co hort =132). For each modeling task, the cohorts were divided into training sets containing ~66% of the cases for variable selection and model calibration, and a held-out testing set containing ~34% of the cases for model validation.

Using only the training set for model development, variables were initially ranked by univariate T-test and Kruskall-Wallis values for diagnosing CAV and for predicting future CAV. Features with p-values < 0.05 were then included in multivariate logistic regression models, with model optimization occurring via backwards elimination. Recognizing that univariate ranking of features can result in excluding variables with potentially important predictive value when considered in the context of multivariable models [Heinze, G. and D. Dunkler, Five myths about variable selection. Transplant International, 2017. 30(1): p. 6-10], vari ables initially excluded due to higher p-values were reevaluated via forward stepwise selection based on achieving a multivariate p value <0.165 (a slightly lenient approximation of the Akaike Information Criteria) [Henize, cited above]; van Smeden, M., et al., Sample size for binary logistic prediction models: Beyond events per variable criteria. Statistical Methods in Medical Research, 2018. 28(8): p. 2455-2474]. After model optimization, leave-one-out cross-validation (LOOCV) was employed to estimate model performance via accuracy and area under the receiver operator characteristic curve (AUROC), and to assess degree of overfit within the training set [Ariel, L., LOOCLASS: Stata module for generating classification statistics of Leave-One-Out cross-validation for binary outcomes. 2015, Boston College Department of Economics]. Selection of the optimal classification cutoff points for accuracy testing within the training sets were guided by the Liu method [Liu, X., Classification accuracy and cut point selection. Stat Med, 2012. 31(23): p. 2676-86]. After locking down final model parameters for study histologic models, the models were next applied to the held-out test sets, with model performance assessed via AUROC, accuracy, sensitivity, and specificity. All statistical analyses were performed in Stata v.15.0 (StataCorp, LLC).

Clinical Risk Factor Modeling: Comprehensive clinical phenotyping of all n=302 patients was performed to enable creation of a ‘Clinical Prediction Model’ (ClinCAV-Pr) for differentiating PrE-CAV patients from No-CAV patients based on baseline and 1-year post transplant patient data. Clinical data included detailed donor and recipient medical history, data pertaining to the transplant event, pre- and post-transplant clinical immunology data, and post-transplant outcomes data including standard histologic assessments of surveillance allograft biopsies. A comprehensive list of captured elements is available in the table with Biologic Bin Definitions/Boundaries. In total, 85 clinical variables were available for predictive modeling. As described with the morphologic data above, the clinical data cohort was divided into a training set containing approximately two-thirds of the cases (n=207), and a held-out testing set containing one third (n=95). To prevent data leakage in the combined model, cases contributing EMB tissue for morphologic analysis were assigned to the same group (training vs testing) as they were for generating and validating the HistoCAV-Pr Model. Variable selection for ClinCAV-Pr was informed by a recent publication which validated six multivariate risk factors for CAV (donor age, donor sex, donor cigarette use, recipient LDL cholesterol at 1-year post transplant, presence of +DSA at 1-year, and history of cellular rejection at 1-year) [Loupy, cited above, 2020], utilizing bidirectional step-wise selection from this six-variable starting point, with variables kept in the model if they achieved a multivariable p-value <0.165 (as described above). As described above, LOOCV was performed to assess ClinCAV-Pr performance in the training set and to optimize classification cutoff points prior to deploying in the held-out test set. The test sets for ClinCAV-Pr and HistoCAV-Pr were congruent, enabling generation of a final iCAV-Pr model that can undergo fair statistical performance comparisons with the baseline models.

Integrated Prediction of Early CAV: The incremental benefit of combining clinical risk factors and morphologic biomarkers was assessed in two ways: 1) via simple, two variable logistic regression using the classification probability outputs from ClinCAV-Pr and HistoCAV-Pr mod els, and 2) by performing backwards elimination and logistic regression using the combined 17 variables from the two modeling approaches. In order to permit such an analysis (and to permit comparisons of ClinCAV-Pr and HistoCAV-Pr models), the test set for validating HistoCAV-Pr was congruent with the larger test set from ClinCAV-Pr validation. The performance of the two modeling approaches for differentiating No-CAV patients from PrE-CAV patients in the training set were compared, with the model with higher averaged AUROC and accuracy on LOOCV being selected as the final ‘histo- clinical’ Integrated CAV prediction model (iCAV-Pr) for validation testing in the held-out test set.

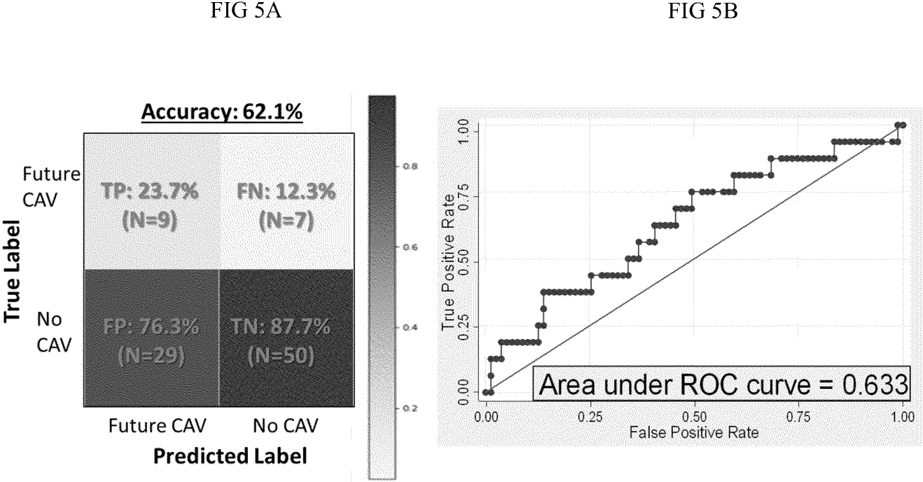

Pre-specified Secondary Analyses: Several secondary analyses were performed to further characterize and contextualized performance of study models. As described in the ‘Clinical Risk Factor Modeling’ section above, six predictive risk factors which had demonstrated multivariable significance in a recent large, multicenter CAV study served as the starting point for stepwise ClinCav-Pr development. In order to permit comparison of previously identified risk factors to this study’s optimized ClinCAV-Pr, we trained and tested the performance of an additional clinical model incorporating just these six validated risk factors within our study cohort FIGS. 5A and 5B. For the HistoCAV-Pr model, a supplemental analysis comparing classification error rates by the ISHLT cellular rejection grade were performed as shown in the following table:. PrE -CAV = pre-early cardiac allograft vasculopathy (CAV) patients, defined as patients experiencing overt CAV by 5-years post-transplant but without diagnosis at 1-year. No-CAV = patients without overt CAV at 6-years post-transplant. ISHLT = international society for heart and lung transplantation.

HistoCAV-Pr Model Performance by ISHLT Grade: Recognizing that there was a baseline imbalance between No-CAV and PrE-CAV groups in the proportion of EMBs with 0R vs. 1R rejection grades (no-CAV: 57.8% 1R grade, while PrE-CAV 76% 1R grade), we explored whether model predictions were strongly associated with underlying ISHLT grade. The Table below shows that misclassifications by the HistoCAV-Pr Model were overall fairly balanced between grades, with a 13.3% (8/60) misclassification rate for 0R EMBs, and a 16.7% misclassification rate for 1R EMBs (p=0.59). Looking more closely, no ISHLT grade does not significantly impact misclassification rates within or between the No-CAV and PrE-CAV groups. This suggests that model performance is not significantly affected by underlying ISHLT grade, and that misclassified cases are unlikely to be the result of the underlying ISHLT grade.

Assessing the impact of differences in the underlying ISHLT grade of study biopsies used for future CAV prediction with the HistoCAV-Pr Model.

| | % ISHLT Grade 0R biopsies Misclassified (n) | % ISHLT Grade 1R biopsies Misclassified (n) | p val (0R vs 1R) |
|---|---|---|---|
| No-CAV | 10.4% (5/48) | 17.6 (6/34) | 0.34 |
| PrE-CAV | 25% (3/12) | 15.8 (6/38) | 0.47 |
| p val (No-CAV vs PrE-CAV) | 0.18 | 0.83 | --- |

Additionally, a comparison of Histo- CAV-Dx and HistoCAV-Pr model performance on slides scanned with each of the two different slide scanning machines used in this study was performed to assess resilience of the feature extraction pipeline to variations in lab equipment.

Morphologic Model Performance by Slide Scanner Manufacturer: While slide scanners are commonplace in pathology departments across the world, there are several different companies manufacturing these devices. Different manufacturers utilize different methods to create digital images, with differences in hardware (charge-coupled device chips vs. lighting bulbs) and software approaches (stitching vs. compression) potentially affecting final image appearance [Janowczyk, A., A. Basavanhally, and A. Madabhushi, Stain Normalization using Sparse AutoEncoders (StaNoSA): Application to digital pathology. Computerized Medical Imaging and Graphics, 2017. 57: p. 50-61]. Prior literature has shown that these differences can potentially impact downstream image analysis pipeline performance. Therefore, we deliberately incorporated digitized slides from two different slide scanner devices in our study to permit an assessment of the resilience of the image analysis pipeline and subsequent models to variations in slide scanner technology. Digital images arising from each scanner were combined into a single cohort prior to randomization to training/test sets. As shown in Table (Performance of study Morphologic Models by slide scanner manufacturer), the overall classification performance (combining training and test set results) of both the HistoCAV-Dx and HistoCAV-Pr Models were unaffected by slide scanner manufacturer. Note that because training and test sets are combined for this analysis, the overall AUROC and accuracy are designed to reflect only overall differences in performance between slide scanners, and not overall validated model performance.

Performance of study Morphologic Models by slide scanner manufacturer.

| | Hamatsu S360 | | Aperio ScanScope | | |
|---|---|---|---|---|---|
| Morphologic Model | | | | | p value |
| | AUROC | Accuracy (n) | AUROC | Accuracy (n) | |
| HistoCAV-Dx Model | 0.93 | 88.3% (68/77) | 0.94 | 89.3% (50/56) | 0.86 |
| HistoCAV-Pr Model | 0.88 | 84.8% (67/79) | 0.90 | 84.9% (45/53) | 0.99 |

### Results

Morphologic Diagnostic Model for CAV: No-CAV vs. Disease Controls:

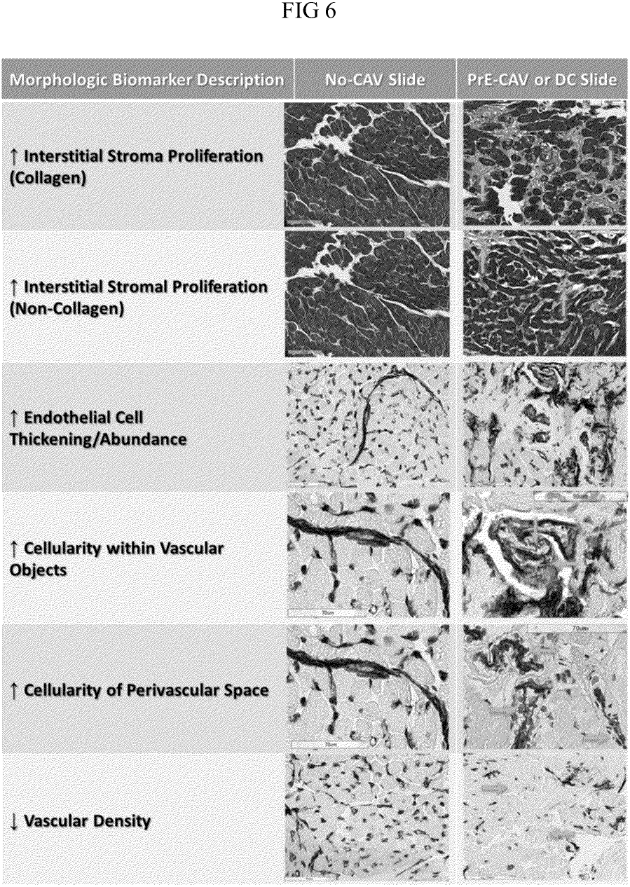

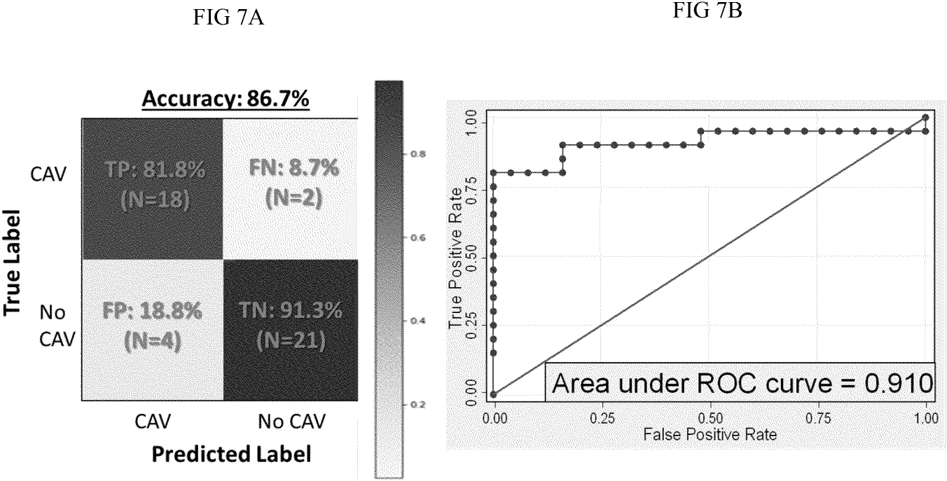

After feature selection as described above, the final HistoCAV-Dx model for discriminating DC tissue samples (obtained at the time of definitive CAV diagnosis) from No-CAV EMB samples (obtained at 1-year post-transplant) incorporated 11 variables. These included three morphologic biomarkers describing stromal ― and in particular, collagen ― proliferation both within and outside the myocardial compartment, one describing decreased overall vascular density of the tissue, five describing the distribution of total nuclei within vs. outside the microvessels, and two describing increased cellular density in the perivascular space of larger capillaries and small pre-capillary arterioles (~25-95 µm²). FIG. 6 provides visual examples of different classes of morphologic biomarkers, while specific model variables with multivariable p values, odds ratios, and annotated variable descriptions are presented in Table (Variables in HistoCAV-Dx Model for Diagnosing cardiac allograft vasculopathy, with annotated descriptions.) As shown in FIGS. 7A and 7B, the HistoCAV-Dx Model for CAV achieved excellent performance after LOOCV in the training set, with an AUROC of 0.91 and an overall accuracy of nearly 88%. On the held-out test set, there was minimal decrement in performance, with an identical AUROC of 0.91 and an accuracy of 86.7%. This suggests sufficient model training, leading to good generalizability without evidence of notable over-fitting.

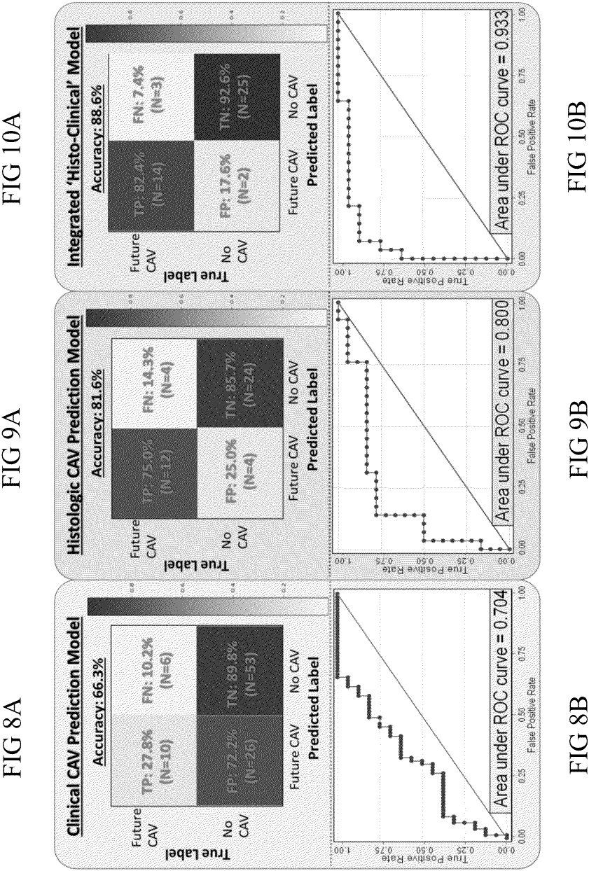

Clinical Risk Factor Modeling: ClinCAV-Pr, for predicting PrE-CAV, included seven clinical risk factors: actively treated recipient diabetes at 1-year (p=0.071), recipient body mass index at one-year post transplant (p=0.087), recipient low-density lipoprotein at one year (p=0.005), a history of high-grade cellular rejection or treated rejection in first year (p=0.16), the percentage of biopsies in the first year with Quilty lesion (p=0.059), donor proteinuria (p=0.018), and donor coronary angiography score (p=0.006). It should be noted that donor coronary angiography score, which is a variable derived from the validated EuroTransplant Heart Donor Score [Smits, J.M., et al., Donor scoring system for heart transplantation and the impact on patient survival. J Heart Lung Transplant, 2012. 31(4): p. 387-97], incorporates and is partially co-linear with donor age, which has consistently been found to be a risk factor for CAV in prior research [Loupy, et al, 2020; Sato, T., et al, Risk Stratification for Cardiac Allograft Vasculopathy in Heart Transplant Recipients- Annual Intravascular Ultrasound Evaluation-. Circulation Journal, 2016.80(2):p.395-403; Nagji, AS. et al, Donor age is associated with chronic allograft vasculopathy after adult heart transplantation: implications for donor allocation. The Annals of thoracic surgery, 2010.90(1): p. 168-175]. Model performance after cross validation is summarized in FIGS. 8A ― 10B. Overall, the clinical model for predicting future development of PrE-CAV achieves modest performance on LOOCV in the training set (AUC 0.745, accuracy 70.5%), with similar (albeit slightly worse) in the held-out test set (AUC of 0.705, accuracy of 66.3%). Positive predictive value is evidently poor at 34% in the training set and 28% in the test set. The apparently strong true-negative rate derives largely from an imbalanced cohort, and overall sensitivity and specificity are both limited at 63% and 67% respectively in the test set.

Morphologic Predictive Model for CAV: No-CAV vs. Early CAV biopsies: The HistoCAV-Pr Model for discriminating between 1-year post-transplant EMB samples from PrE-CAV patients vs. No- CAV patients incorporated 10 variables. These included morphologic biomarkers describing (non-collagen) stromal proliferation, total microvessel-staining-area-to-myocyte-staining area ratio, the proportion of total microvessel count/area accounted for by microvessels of different sizes (with results suggesting a decrease in smaller microvessel density and an increase in relative staining area of larger microvessels), and a variable characterizing the cellular density in the perivascular spaces surrounding pre-capillary arterioles (~65-500 µm²). Visual examples of different morphologic biomarker classes are presented in FIG. 6, and specific HistoCAV-Pr variables with multivariable p values, odds ratios, and annotated variable descriptions are presented in Table entitled Variables in HistoCAV-Pr for predicting future cardiac allograft vasculopathy, with annotated descriptions, and representative examples of No-CAV and PrE-CAV slides can be examined for qualitative differences in microarchitecture by referring back to FIG. 1. As shown in FIGS. 10A-10B, the HistoCAV-Pr Model for PrE-CAV prediction achieved good performance on LOOCV in the training set, with an AUROC of 0.864 and an accuracy of 80.8%. In the held-out test set, model performance was similar, with an AUROC of 0.80 and an accuracy of 81.6%. Overall, this performance represents a clear improvement over ClinCAV-Pr in terms of overall performance, with a marked improvement in positive predictive value (75% vs. 27%,, p=0.016).

Integrated CAV Prediction: The final integrated iCAV-Pr model for PrE-CAV prediction selected for validation testing was a simple, 2-variable logistic model incorporating the prediction probabilities of ClinCAV-Pr and HIstoCAV-Pr models. This model achieved excellent predictive performance after LOOCV in the training set, with an AUROC of 0.939 with an accuracy of 87.5%. Performance of the final iCAV-Pr model was essentially identical in the held-out test set as compared to the training set, with an AUROC of 0.933 and an accuracy of 88.6%, suggesting reasonable model saturation during training and good generalizability. As shown in FIG. 5, the iCAV-Pr model demonstrated clear, incremental improvement in AUROC and accuracy within the same training and test cases when compared to either constituent model. Statistical comparison of accuracy and AUROC results unambiguously confirms that the addition of morphologic biomarkers adds significant improvements in CAV prediction over traditional clinical risk factors (p=0.005 for accuracy, p=0.009 for AUROC). Interestingly, the final iCAV-Pr model using just the predictive probabilities of ClinCAV-Pr and HistoCAV-Pr as model inputs outperformed a model developed via back- wards-elimination using all 17 variables from the final ClinCav-Pr and HistoCAV-Pr models (this 12 variable model achieved an AUROC of 0.87 and an accuracy of 85% after LOOCV.

### Discussion

We have presented compelling findings herein supporting the utility of EMB tissue analysis for diagnosing and predicting CAV. The quantitative histologic analysis method deployed in his work permitted not only the characterization of morphologic biomarkers associated with advanced CAV, but also the discovery of novel biomarkers which are present in EMB samples years before overt CAV development. These predictive morphologic biomarkers were not only superior to traditional clinical risk factors, but were also orthogonal to them, enabling superior CAV risk assessments when morphologic predictors and clinical predictors are combined.

The present work deploys computer assisted histology workflows within cardiovascular medicine. Recent efforts have focused on diagnostic pipelines, evaluating cardiac tissue in order to better describe disease states as they currently exist. Specifically within cardiac transplant, several studies have used quantitative analyses to examine transplant EMBs in recent years. However, these works largely focused on acute rejection grading, either trying to develop automated systems to complete this task more reliably [Peyster, E.G., et al., An automated computational image analysis pipeline for histological grading of cardiac allograft rejection. European Heart Journal, 2021. 42(24): p. 2356-2369; Dooley, A.E., et al., Prediction of Heart Transplant Rejection Using Histopathological Whole-Slide Imaging. IEEE EMBS Int Conf Biomed Health Inform, 2018. 2018], or piloting more expensive and complex in-situ analyses to aid in discerning more benign vs. more serious immune cell infiltrates [Peyster, E.G., et al., In Situ Immune Profiling of Heart Transplant Biopsies Improves Diagnostic Accuracy and Rejection Risk Stratification. JACC: Basic to Translational Science, 2020. 5(4): p. 328-340]. This work differs from these prior efforts in its focus on using clinical EMB samples to predict a distant future outcome.

Prior research in cardiac digital pathology involved both ‘deep learning’ methods which prioritize model performance rather than model interpretability [Nirschl, J.J., et al., A deep-learning classifier identifies patients with clinical heart failure using whole-slide images of H&E tissue. PLOS ONE, 2018. 13(4): p. e0192726; Nirschl, J., et al., Deep Learning Tissue Segmentation in Cardiac Histopathology Images. 2017. 179-195], as well as ‘hand-crafted’ methods which utilize supervised feature extraction in order to provide both performance and explanation [Peyster, E.G., et al., An automated computational image analysis pipeline for histological grading of cardiac allograft rejection. European Heart Journal, 2021. 42(24): p. 2356-2369]. These examples rely on a hand-crafted approach, and while this may come at a small decrement in model performance as compared to deep learning methods, the reliance on explicitly defined and demonstrable morphologic features both improves model acceptance and also enables a glimpse into underlying biology that cannot be elucidated using more opaque deep learning methods. For example, the importance in both morphologic models of image features describing the cellularity of the perivascular space highlights around pre-capillary arterioles (~65-500 µm²) highlights an area for deeper mech anistic study. While endothelitis and endothelial hyperplasia/proliferation are recognized as histologic features of CAV, our results suggest that they are both early (occurring as soon as 1-year post transplant) and persistent finding (present at the time of CAV diagnosis) findings. Investigations exploring the identity, function, and movement of these cells may lead to a more comprehensive understanding of CAV pathogenesis, as well as new strategies for preventing and interrupt ing its development. Additionally, further study into the perivascular cellular environment may have implications for other diseases thought to involve cardiac microvascular dysfunction, such as diabetes or heart failure with preserved ejection fraction.

From a clinical perspective, though CAV is a disease with existing treatment and prevention strategies, the application of these strategies lack both timeliness and precision. Thus, a key unmet need in CAV mitigation is personalized risk-stratifi cation which can confidently describe where a patient will be rather than where they are right now. A recent prospective observational study attempted to address this need using a large, international cohort for modeling patient CAV trajecto ries [Loupy et al, 2020, cited above]. While the scale and rigor of that work is commendable, reliance on a relatively small set of traditional clinical varia bles (six total donor or recipient risk factors) has raised legitimate concerns over whether such an approach can truly provide confident predictions at the individual patient level [Moayedi, Y. and J.J. Teuteberg, Predicting Where Patients Will Be, Rather Than Just Seeing Where They Are. Circulation, 2020. 141(24): p. 1968-1970]. These concerns arise not from the excellent false negative rate of their CAV trajectory model, but rather from the relatively low confidence with which the model predicts clinically serious CAV trajectories [Moaydedi, cited above]. In the present experiment, both the final ClinCAV-Pr model and the clinical dataset from which it is derived have substantial overlap with the risk factors used in this prior CAV trajectory study. Indeed, our results con firm their fundamental findings - that statistical modeling of donor/recipient cardiovascular risk factors and early post- transplant evidence of allo-immune activity has predictive value. However, our results also validate the aforementioned concerns regarding high false-positive rates when relying on clinical variables alone for CAV risk prediction. If models like the ClinCAV-Pr or the 6-variable model we developed using the prior publication’s validated risk factors (which had even poorer statistical performance) were deployed clinically, this could translate to a substantial iatrogenic harm through es calations in immunosuppression and invasive surveillance for patients who are unlikely to benefit. Thus, the clear strength of the present research is in recognizing the value of broad clinical risk factors, while also acknowledging the need to look beyond traditional risk factors via advanced morphologic analysis of routinely acquired EMB tissues. The end result of this integrated approach is a predictive assay that looks deeply at tissue, but which also contextualizes the fine-detail of tissue- level findings with pre-test probabilities determined through patient-level risk factors. While conceptually intuitive, the integrative approach employed in this manuscript represents a novel and promising paradigm within cardiovascular (and transplant) medicine.

Despite the strong performance of the study histologic models, misclassifications still occurred in each experiment. An ticipating that some degree of misclassification was inevitable, we pre-specified several analyses designed to explore po tential contributing factors. Specifically, we conducted analyses to test whether the ISHLT grade, the presence of Quilty, or the presence of prior biopsy site artifacts might affect model performance. These analyses (provided in the supplement) show no significant differences in traditional histology parameters between misclassified and correctly classified EMBs, providing no clear explanation for misclassifications and instead suggesting that the histologic models function in a largely ‘grade-agnostic’ fashion. Interestingly, ClinCAV-Pr performed essentially as expected on cases incorrectly classified by the HistoCAV-Pr Model, achieving an accuracy of 65% and suggesting that clinical risk factors were not substantially different in the misclassified group. It is possible that tissue or stain quality impacted classification performance, with slides that were outliers in certain aspects of slide quality contributing to classification errors. If this were the case, it would represent a problem that could potentially be solved by increasing the size of the training set to enable the model to be calibrated on a broader spectrum of tissue/stain quality. Beyond issues of slide quality, it is also possible that some misclassifications are the result of factors that have nothing to do with model bias or model training. Sampling error is a recognized issue in all histologic fields, and certainly could contribute to mis- classification through random sampling of a region of tissue that does not reflect the majority of the myocardium. Additionally, it is worth considering that the types of allo-immunity that lead to CAV are not static processes, and the progres sion from healthy myocardium to diseased myocardium is neither a continuous process nor a process with a standardized starting time. Patients with evidence of early tissue damage and ongoing allo-immunity at 1-year may sometimes stabilize with adjustments in immunosuppression, improvements in compliance, or the development of ‘accommodation’ in the allograft [Colvin, M.M., et al., Antibody-Mediated Rejection in Cardiac Transplantation: Emerging Knowledge in Diagnosis and Management. Circulation, 2015. 131(18): p. 1608-1639]. On the other hand, patients who do not manifest significant allo-immunity in the first year may experience changes reductions in immunosuppression or sensitizing events that predispose them to later allo-immune activation and consequent development of CAV. For these reasons, ‘perfect’ prediction at one year may be not actually be possible from analysis of the cardiac tissue alone.

As with all experiments, the findings reported in this manuscript should be interpreted in the context of the study’s limitations. The most prominent among these is the size and scope of the study cohort. As a modestly sized, single-center investigation, the generalizability of study models remains unsettled. While strong validation results on held-out test sets, along with consistent performance across two different scanning devices and two different slide-processing batches pro- vides some confidence in the generalizability of the pipeline, further external (and, ideally, prospective) validation represents an important next step on the path to clinical translation. Such external validation efforts should focus not only on including data from multiple centers, but would ideally also incorporate additional forms of CAV diagnostic testing such as Intravascular ultrasound. While angiography with intravascular ultrasound is invasive and technically challenging, it can both aid in contextualizing our morphologic biomarkers and in identifying the best populations (and timing) for incorporating digital pathology versus invasive angiographic CAV screening approaches. It should also be noted that while this study utilized EMB tissues as already collected during routine clinical care, the staining for CD31 and Movats pentachrome are not necessarily routine staining for clinical EMB samples. Whether a quantitative histology pipeline for CAV which utilizes only routine H&E stained slides would perform as well remains unsettled. If additional stains are indeed required for best predictive results, then the added time and expense of slide processing would have to be considered an additional challenge for clinical translation, though it should be noted that this one-time expense on 1-year post-transplant biopsies is not substantial (and may be offset by the reduced frequency of stress testing and angiography for low-risk patients).

In conclusion, we demonstrate that advanced digital pathology methods can identify early microvascular manifestations of CAV with high sensitivity and specificity. Moreover, a model which combines digital pathology ‘morphologic biomarkers’ with conventional clinical risk factors enables the identification of patients who will develop aggressive CAV years before overt disease onset. If replicated in an external cohort, our integrated approach to CAV prediction could open a new frontier in personalized care for heart transplant recipients, with the potential to tailor both screening and preventative therapeutic strategies to individual CAV risk profiles.

All publications cited in this specification, including those specifically recited above, are incorporated herein by reference.

While the principles of various embodiments of the invention(s) have been described above in connection with specific devices, apparatus, systems, algorithms, and/or methods, it is to be clearly understood that this description is made only by way of example and not as limitation. One of ordinary skill in the art will appreciate that various modifications and changes can be made without departing from the scope of the claims below.

The above description illustrates various embodiments along with examples of how aspects of particular embodiments may be implemented, and are presented to illustrate the flexibility and advantages of particular embodiments as defined by the following claims, and should not be deemed to be the only embodiments. One of ordinary skill in the art will appreciate that based on the above disclosure and the following claims, other arrangements, embodiments, implementations and equivalents may be employed without departing from the scope hereof as defined by the claims. Accordingly, the specification and figures are to be regarded in an illustrative rather than a restrictive sense, and all such modifications are intended to be included within the scope of the present invention. The benefits, advantages, solutions to problems, and any element(s) that may cause any benefit, advantage, or solution to occur or become more pronounced are not to be construed as a critical, required, or essential features or elements of any or all the claims.

1. A method for diagnosing, predicting and/or preventing acute allograft vasculopathy in an organ transplant patient, the method comprising executing on a processor the steps of:
(a) analyzing clinical risk factors of a cardiac, kidney or liver transplant patient in a model which distinguishes between clinical biomarkers of allograft vasculopathy patients and non-allograft vasculopathy patients, and
(b) analyzing digital biopsy images from the transplant patient to morphologically differentiate patients which are prE allograft vasculopathy or which have active allograft vasculopathy from patients without acute allograft vasculopathy;
(d) assigning prE-, active or non- allograft vasculopathy status to a patient based on the outcome of (a) and (b); and
(e) where preE or active statis is assigned, treating the patient.

2. The method according to claim 1, wherein the digital biopsy images comprise at least one stain specific for endothelial cells and/or connective tissues of the vascular cells and tissues.

3. The method according to claim 2, wherein the stain is selected from CD31, CD34, CD68, or combinations thereof.

4. The method according to claim 2, wherein the stain is a connective tissue stain selected from Mason’s trichrome or Movats pentachrome stain.

5. The method according to claim 1, wherein the patient is a cardiac transplant patient and the digital biopsy images are from an endomyocardial biopsy (EMB) obtained at about 1 year post-transplant.

6. The method according to claim 5, wherein the patient is assigned prE or active cardiac allograft vasculopathy status.

7. The method according to claim 1, wherein the analysis of morphologic biomarkers in (b) and (c) comprises analysis of (i) interstitial composition and proliferation, (ii) myocyte density, (iii) total microvascular density, (iv) the proportions of vessels of different sizes, and (v) the cellular abundance both within microvessels and in the immediate perivascular space around these vessels.

8. The method according to claim 1, wherein the patient is assigned positive status for pre-acute vasculopathy or active vasculopathy if the biomarkers is present: increased overall stromal content area in a connective tissue stained slide; increased interstitial collagen content; increased non-collagen interstitial stromal content; increased thickness of staining of individual vascular structures; increased cellularity within stained vascular structures; increased cellularity in the perivascular area immediately surrounding stained vascular structures; and/or decreased overall tissue-density of vascular structures.

9. The method according to claim 1 for use in diagnosing active cardiac allograft vasculopathy, wherein analyzing (b) comprises analyzing: percentage of total vascular nuclei found within larger capillaries and small pre-capillary arterioles; number Nuclei within vasculature as normalized by myocyte area; number of Nuclei outside the vascular/perivascular space as normalized by myocyte area; percentage of total nuclei in perivascular space as normalized by myocyte area; number of Nuclei within in vasculature as normalized by stromal area; number of Nuclei outside the vasculature/perivascular space as normalized by stromal area; Total stroma to myocardium ratio; Collagen content of interstitium; percentage of vascular nuclei around medium microvessels as normalized by stromal area, and Total vascular density as determined by vascular object area to myocardium area; and Interstitial stromal area.

10. The method according to claim 1 for use in predicating acute allograft vasculopathy, wherein (b) comprises analyzing: Interstitial stroma as normalized by myocardial compartment size; percentage of microvessels that are larger capillaries and small pre-capillary arterioles; Non-collagen/non-proteoglycan content of interstitium; number of perivascular nuclei per vessel area for pre-capillary arterioles; total stroma to myocardium ratio; percentage of total DAB/vascular area comprised of pre-capillary arterioles; total microvascular staining area as normalized by myocardium; microvascular staining area of pre-capillary arterioles as normalized; total # of nuclei in and around vessels as normalized by myocardium; and percentage of total vascular nuclei found within capillaries.

11. The method according to claim 9, wherein ‘pre-capillary arterioles’ are about 65 µm² to about 500 µm² and ‘larger capillaries and small pre-capillary arterioles’ are below about 25 µm² to about 95 µm².

12. The method according to claim 1, wherein a prE-CAV patient is assigned wherein the computer-implemented analysis of the digital EMB identifies one or more of:
increased overall stromal content area in a Movats pentachrome stained slide;
increased interstitial collagen content;
increased non-collagen interstitial stromal content;
increased CD31 staining of vascular structures;
increased cellularity within CD31 vascular structures;
increased cellularity in the perivascular area immediately surrounding CD31 vascular structures; and/or
area of decreased overall vascular density.

13. The method according to claim 1 wherein the clinical risk factors comprise: (i) actively treated recipient diabetes at one-year post-transplant, (ii) recipient body mass index at one-year post transplant, (iii) recipient low-density lipoprotein at one year post-transplant, (iv) a history of high-grade cellular rejection or treated rejection in first year, (v) the percentage of biopsies in the first year with Quilty lesion, (vi) donor proteinuria, and/or (vi) donor coronary angiography score.

14. The method according to claim 1, wherein the method comprises analyzing in (a) History of Cellular Rejection, Recipient BMI, Donor Coronary Angiography Score, and Recipient Diabetes and analyzing in (b) Interstitial stroma as normalized by myocardial compartment size; Non-collagen/non-proteoglycan content of interstitium; number of perivascular nuclei per vessel area for pre-capillary arterioles; Total stroma to myocardium ratio; Total microvascular staining area as normalized by myocardium; Microvascular staining area of pre-capillary arterioles as normalized; Total number of nuclei in and around vessels as normalized by myocard, and percentage of total vascular nuclei found within capillaries.

15. The method according to claim 1, wherein the method further comprises, prior to (b):
obtaining a biopsy;
staining tissues from the biopsy with CD31 and generating digital images; and
staining tissues from the biopsy with Movats and generating digital images.

16. The method according to claim 1, wherein a positive patient is treated with a regimen comprising: one or more immunosuppressants, one or more of a statin, mycophenolate mofetil, everolimus, sirolimus, aspirin, vitamins, a PCSK-9 inhibitor, a P2y12 inhibitor, ezetimibe, and/or fish oil or.

17. The method according to claim 1, wherein the patient is a kidney transplant patient.

18. A computer-implemented method for detection of a pre-CAV or active CAV patient, comprising executing on a processor the steps of:
(a) analyzing clinical risk factors of a cardiac transplant patient in a model which distinguishes between clinical biomarkers of (i) prE-CAV or active CAV patient and (ii) non-prE-CAV or non-CAV patients, and
(b) analyzing digital endomyocardial biopsy (EMB) images from the cardiac transplant patient to morphologically differentiate (i) PrE-CAV or active CAV from (ii) non-prE-CAV or non-CAV patients; and
(c) assigning (i) PrE-CAV or active CAV or (ii) non-PrE-CAV or non-CAV status to a patient.

19. A non-transitory computer-readable storage medium comprising stored instructions which, when executed by one or more computer processors, cause the one or more computer processors to perform steps of:
(a) analyzing clinical risk factors of a cardiac transplant patient in a model which distinguishes between clinical biomarkers of (i) prE-CAV or active CAV and (ii) non-prE-CAV or non-CAV patients, and
(b) analyzing digital endomyocardial biopsy (EMB) images from the cardiac transplant patient to morphologically differentiate (i) PrE-CAV or active CAV from (ii) non-prE-CAV patients; and
(c) assigning PrE-CAV or non-PrE-CAV status to a patient.

20. A system for detection of a pre-CAV patient, comprising at least one processor configured to perform steps of:
(a) analyzing clinical risk factors of a cardiac transplant patient in a model which distinguishes between clinical biomarkers of (i) prE-CAV or active CAV patients and (ii) non-prE-CA V or non-CAV patients, and
(b) analyzing digital endomyocardial biopsy (EMB) images from the cardiac transplant patient to morphologically differentiate (i) prE-CAV or active CAV patients and (ii) non-prE-CAV or non-CAV patients; said at least one processor being configured to PrE-CAV or non-PrE-CAV status to a patient.
